SEQUENCE LISTING (11 sequences)

SEQ ID NO: 1 - protein, 322 aa, Scheffersomyces xylosifermentans
MVAPIPQIKLSSGHLMPSIGFGCWKLDKPTAGEQVYEAIKAGYRLFDGAEDYGNEKEIGDGVKKAIDEGIVKREELFLVSKLWNNYHDPKNVETALDRTLADLKTDYVDLFYIHFPIAFKFVPLEEKYPPAFYCGDGDNFHYEDVPILETWKALEKLVASGKIRSIGVSNFPGALLLDLLRGATIKPAVLQVEHHPYLQQPRLIEFAQKAGLTVTAYSSFGPQSFVEMNQGRALKTPPLFENEVIKAIAAKHNRAPAEVLLRWSAQRGIAVIPKSNLPERLVQNRGFNSFDLTKEDFEEIAKLDINLRFNDPWDWDHIPIFV

SEQ ID NO: 2 - DNA, 969 bp, Scheffersomyces xylosifermentans
  source 1..969: mol_type = genomic DNA
atggttgccccaatcccacaaatcaaattgagctccggtcacttgatgccttccatcggtttcggctgttggaagctcgacaagccaaccgccggtgaacaagtctacgaagccatcaaggccggttacagattgttcgacggtgctgaggactatggtaacgaaaaggaaatcggtgacggtgtcaagaaggctattgatgaaggtatcgtcaagagagaagagctcttccttgtctccaagttgtggaacaactaccacgacccaaagaacgtcgagactgccttggacagaaccctcgctgaccttaagaccgactacgttgacttgttctatatccacttcccaattgccttcaagttcgtcccactcgaggagaagtacccaccagcattctactgtggtgacggtgacaacttccactacgaagacgtcccaatcttggagacctggaaggccctcgagaagttggtcgcttccggtaagattagatccatcggtgtctccaacttcccaggtgctttgctcttggacttgctcagaggtgctaccatcaagccagctgttttgcaagttgagcaccacccatacttgcaacaaccaagattgattgagttcgcccaaaaggccggtcttaccgtcactgcttactcttctttcggtcctcaatccttcgttgagatgaaccaaggtagagctttgaaaaccccaccattgttcgaaaacgaggtcatcaaggctattgctgccaagcacaacagggccccagctgaggttttgttgagatggtctgctcaaagaggtattgctgtcattccaaagtctaacctcccagagagattggtccaaaacagaggtttcaacagcttcgacttgaccaaggaggacttcgaggagatcgccaagttggacatcaacttgagattcaacgacccatgggactgggaccatatcccaatcttcgtttaa

SEQ ID NO: 3 - protein, 364 aa, Scheffersomyces xylosifermentans
MTANPSLVLNKIDDITFETYDAPEITEPTDVLVEVKKTGICGSDIHYYAHGKIGNFVLTKPMVLGHESSGVVTKIGKGVTSLKVGDKVAIEPGIPSRFSDAYKSGHYNLCPHMCFAATPNSTEGEPNPPGTLCKYFKSPEDFLVKLPEHVSLEMGALVEPLSVGVHASKLASVRFGDFVAVFGAGPVGLLAAAVAKTFGAKGVIVIDIFDNKLQMAKDIGAATHIFNSKTGGDAAALIKAFDGHEPTVVLECTGAEPCINQGVAILAQGGRFVQVGNAPAPVKFPITEFATKELTLFGSFRYGFNDYKTSVDIMDTNYQNGKEKAPIDFEQLITHRFKFDDAIKAYDLVRAGSGAVKCIIDGPE

SEQ ID NO: 4 - DNA, 1095 bp, Scheffersomyces xylosifermentans
  source 1..1095: mol_type = genomic DNA
atgactgccaacccatccctcgttctcaacaagatcgacgacatcaccttcgaaacctacgatgccccagagatcacggaaccaaccgatgttctcgtagaagtcaagaaaacaggtatctgtggttcagatatccactactacgcccacggtaagatcggtaacttcgtgttgaccaagccaatggttcttggccacgaatcctctggtgtcgtcaccaagatcggtaagggtgttacctccctcaaggtcggagacaaggtcgccattgagccaggtattccatccagattcagtgacgcctacaagagcggtcactacaacttatgtccacacatgtgcttcgctgccactccaaactccaccgagggcgagccaaacccaccaggaaccttatgtaagtacttcaagtccccagaggatttcttggtcaagttgccagaacacgtttccttggaaatgggtgctcttgtcgagccattgtctgtcggtgtccacgcttccaagttggcttccgtcagattcggtgattttgtcgctgtcttcggtgctggtccagtcggtctcttagctgctgccgtcgccaagaccttcggtgctaagggtgtcattgtcattgacattttcgacaacaagttgcaaatggccaaggacatcggtgctgctacccacatcttcaactccaagaccggcggtgacgccgctgccttaattaaggctttcgacggccacgagccaaccgtcgttttggaatgtactggtgctgagccatgtatcaaccaaggtgtcgctatcttggcccaaggtggtcgtttcgtccaagtcggtaacgccccagccccagtgaagttcccaatcaccgagttcgctaccaaggaactcaccttgttcggatctttcagatacggtttcaacgattacaagacctctgtcgacatcatggacaccaactaccagaacggtaaggaaaaggccccaattgacttcgaacaattgatcacccacagattcaagttcgacgacgccattaaggcctacgacttggtcagagctggtagtggtgctgtcaagtgtatcattgacggtcctgagtaa

SEQ ID NO: 5 - protein, 628 aa, Scheffersomyces xylosifermentans
MTTSLYDSSELLFLGFDLSTQQLKIIVTNEKLAALKTYNVEFDVLNKDVYGVHKGVIAIDNEEHKGAIVSPVFMWLDSLDQIFEDMKKDSFPFNKVAGVSGSCQQHGSVFWSKQAEAALAELQAKSTLTEQLKNAFTFKHSPNWQDHSTGKELAEFEKIIGADNLADISGSRAHYRFTGLQIRKLATRFNPAKYNATWRISLVSSFLASVLLGKVTTIEEADACGMNLYDIKAGDFNEDLLAIAAGVHPELDNVKKDSPEYVAGVTALREKLGKVRPISYESEGKISPYFVDRYGFNPECQVYSFTGDNLATIISLPLAPNDALISLGTSTTVLIITKNYNPSSQYHLFKHPTMPDHYMGMICYCNGSLAREKVRDEINEKYNVKDTKSWDKFNEILDKSHSFNNKLGIYFPLGEIVPNAAAQTKRSILTAENKIVDVELGEKNWVEDDDVSSIVESQTLSCRLRAGPMLSGSVKTTASTSPSPPPEGDDKQHLHHVYKDLVDKFGELYTDGKKQTYESVTARPNRCYYVGGASNNGSIIRKMGSILAPLNGNYKVEIPNACALGGAYKASWSYECEAKKEWIAYDKYINRLFEKSDGMEKFEAEDKWLDYFTGVGLLAKMESELKHN

SEQ ID NO: 6 - DNA, 1887 bp, Scheffersomyces xylosifermentans
  source 1..1887: mol_type = genomic DNA
atgaccacctctctctacgacagctccgagctgcttttcctcgggttcgatctctcgacccagcagttgaagatcatcgtcaccaacgagaagctcgcagccttgaagacctacaacgtcgagttcgatgtgctcaacaaggacgtctatggcgtccacaagggtgtcattgctattgacaacgaagaacacaagggtgccatcgtttccccagttttcatgtggttggactcgcttgaccagattttcgaggatatgaagaaggattcgttccctttcaacaaggttgctggtgttagtggatcttgtcaacaacacggttctgttttctggtccaaacaagccgaagccgctttggccgaattgcaagctaagtctactttgaccgaacagttgaaaaacgccttcaccttcaagcactctccaaactggcaggaccattccaccggtaaggagttggctgagttcgagaagatcatcggtgccgataacttggccgatatctcgggttccagagcccattaccgtttcaccggtcttcagatcagaaaattggctaccagattcaacccagccaagtataatgctacctggagaatctcattggtgtcttcattcttggccagtgttttgctcggtaaagtcaccaccatcgaagaagcagatgcttgtggtatgaacctttacgatatcaaggctggtgacttcaacgaagacctcttggccatcgctgccggtgtccacccagaattggacaatgtcaagaaagacagtccagaatacgtcgctggtgtaactgctttacgtgaaaagttgggtaaggtccgtcctatctcttacgagagtgaaggtaagatttctccatacttcgtggacagatacggcttcaacccagagtgtcaggtctactctttcactggagacaacttggctaccatcatttcgttacctcttgcaccgaacgatgccttaatctcgttaggtacatccaccacagtgttgattatcaccaaaaactacaacccttcgtctcaataccacttgttcaagcatccaacaatgccagaccactacatgggtatgatctgctactgtaacggttccttggccagagaaaaggtcagagacgaaatcaatgagaaatacaacgtcaaggacaccaagtcttgggacaagttcaacgaaatcttggacaaatcccactctttcaacaacaagttgggtatctacttcccattgggagagattgttcctaatgctgctgcacagaccaagagatctattttgaccgccgaaaataagattgtcgatgtcgaattgggcgaaaagaactgggtggaggatgatgatgtctcctccattgttgagtcccagacattgagttgtagattgagagccggcccaatgttgagtggaagtgtgaagacaactgcatctacttctccttctccaccaccagaaggagacgacaagcaacacttgcaccatgtctacaaggatttggtcgacaaattcggcgagttgtacaccgatggaaagaaacaaacctacgagtccgtcactgccagacctaaccgttgttactacgtcggtggtgcttccaacaacggaagtatcattcgtaagatgggttccattttggcccctctcaatggtaactacaaggtggagattcctaacgcctgtgccttaggtggagcctacaaggccagttggagttacgaatgcgaggccaagaaggaatggatcgcctatgacaagtacatcaaccgtttgtttgagaagagtgacggaatggaaaagttcgaggctgaagacaagtggcttgactacttcactggagttggcttgttagccaagatggagagcgaattaaagcacaactag

SEQ ID NO: 7 - protein, 318 aa, Scheffersomyces stipitis
MPSIKLNSGYDMPAVGFGCWKVDVDTCSEQIYRAIKTGYRLFDGAEDYANEKLVGAGVKKAIDEGIVKREDLFLTSKLWNNYHHPDNVEKALNRTLSDLQVDYVDLFLIHFPVTFKFVPLEEKYPPGFYCGKGDNFDYEDVPILETWKALEKLVKAGKIRSIGVSNFPGALLLDLLRGATIKPSVLQVEHHPYLQQPRLIEFAQSRGIAVTAYSSFGPQSFVELNQGRALNTSPLFENETIKAIAAKHGKSPAQVLLRWSSQRGIAIIPKSNTVPRLLENKDVNSFDLDEQDFADIAKLDINLRFNDPWDWDKIPIFV

SEQ ID NO: 8 - protein, 363 aa, Scheffersomyces stipitis
MTANPSLVLNKIDDISFETYDAPEISEPTDVLVQVKKTGICGSDIHFYAHGRIGNFVLTKPMVLGHESAGTVVQVGKGVTSLKVGDNVAIEPGIPSRFSDEYKSGHYNLCPHMAFAATPNSKEGEPNPPGTLCKYFKSPEDFLVKLPDHVSLELGALVEPLSVGVHASKLGSVAFGDYVAVFGAGPVGLLAAAVAKTFGAKGVIVVDIFDNKLKMAKDIGAATHTFNSKTGGSEELIKAFGGNVPNVVLECTGAEPCIKLGVDAIAPGGRFVQVGNAAGPVSFPITVFAMKELTLFGSFRYGFNDYKTAVGIFDTNYQNGRENAPIDFEQLITHRYKFKDAIEAYDLVRAGKGAVKCLIDGPE

SEQ ID NO: 9 - protein, 623 aa, Scheffersomyces stipitis
MTTTPFDAPDKLFLGFDLSTQQLKIIVTDENLAALKTYNVEFDSINSSVQKGVIAINDEISKGAIISPVYMWLDALDHVFEDMKKDGFPFNKVVGISGSCQQHGSVYWSRTAEKVLSELDAESSLSSQMRSAFTFKHAPNWQDHSTGKELEEFERVIGADALADISGSRAHYRFTGLQIRKLSTRFKPEKYNRTARISLVSSFVASVLLGRITSIEEADACGMNLYDIEKREFNEELLAIAAGVHPELDGVEQDGEIYRAGINELKRKLGPVKPITYESEGDIASYFVTRYGFNPDCKIYSFTGDNLATIISLPLAPNDALISLGTSTTVLIITKNYAPSSQYHLFKHPTMPDHYMGMICYCNGSLAREKVRDEVNEKFNVEDKKSWDKFNEILDKSTDFNNKLGIYFPLGEIVPNAAAQIKRSVLNSKNEIVDVELGDKNWQPEDDVSSIVESQTLSCRLRTGPMLSKSGDSSASSSASPQPEGDGTDLHKVYQDLVKKFGDLYTDGKKQTFESLTARPNRCYYVGGASNNGSIIRKMGSILAPVNGNYKVDIPNACALGGAYKASWSYECEAKKEWIGYDQYINRLFEVSDEMNSFEVKDKWLEYANGVGMLAKMESELKH

SEQ ID NO: 10 - DNA, 20 bp, synthetic construct
  source 1..20: mol_type = other DNA
caacagccaaaacccacggc

SEQ ID NO: 11 - DNA, 20 bp, synthetic construct
  source 1..20: mol_type = other DNA
cttaaccaagaaatcttcgg

(19) United States
(12) Patent Application Publication
(10) Pub. No.: US 2025/0163362 A1
(43) Pub. Date: May 22, 2025

(54) RECOMBINANT YEASTS WITH RECOMBINANT XYLOSE REDUCTASE, XYLITOL DEHYDROGENASE, AND/OR XYLULOKINASE GENES AND METHODS OF USING SAME

(71) Applicant: Wisconsin Alumni Research Foundation, Madison, WI (US)

(72) Inventors: Trey Sato, Madison, WI (US); Christopher Todd Hittinger, Madison, WI (US); Katharina Barros, Madison, WI (US)

(73) Assignee: Wisconsin Alumni Research Foundation, Madison, WI (US)

(21) Appl. No.: 18/956,291

(22) Filed: Nov. 22, 2024

Related U.S. Application Data
(60) Provisional application No. 63/601,821, filed on Nov. 22, 2023.

(51) Int. Cl. C12N 1/18; C12N 9/04; C12N 9/12; C12P 7/06; C12R 1/865

(52) U.S. Cl. CPC C12N 1/18; C12N 9/0006; C12N 9/1205; C12P 7/06; C12R 2001/865; C12Y 101/01009; C12Y 101/01307; C12Y 207/01017

(57) ABSTRACT

Recombinant yeasts comprising recombinant xylose reductase, xylitol dehydrogenase, and/or xylulokinase genes and methods of using same, such as for producing ethanol from xylose-containing feedstocks.

### STATEMENT REGARDING FEDERALLY SPONSORED RESEARCH

This invention was made with government support under 2110403 awarded by the National Science Foundation and under DE-SC0018409 awarded by the US Department of Energy and under 23-CRHF-0-6055, 24-CRHF-0-6055 awarded by the USDA/NIFA. The government has certain rights in the invention.

### SEQUENCE LISTING

The instant application contains a Sequence Listing which has been submitted in XML format and is hereby incorporated by reference in its entirety. The XML copy, created on Sep. 13, 2024, is named USPTO--09824556-P240092US02--SEQ_LIST.xml and is 17,093 bytes in size.

### FIELD OF THE INVENTION

The invention is directed to recombinant yeasts comprising recombinant xylose reductase, xylitol dehydrogenase, and/or xylulokinase genes and methods of using same, such as for producing ethanol from xylose-containing feedstocks.

### BACKGROUND

Xylose is the most abundant pentose comprised by hemicellulose in plants. Robust microorganisms that can ferment this sugar are therefore required for profitable biofuel production from lignocellulosic materials (1,2). The budding yeast Saccharomyces cerevisiae is widely used in biotechnological applications and is one of the most understood model microorganisms (3,4). However, S. cerevisiae lacks certain traits that limit its usefulness in lignocellulosic biofuel production. For example, S. cerevisiae does not have the ability to ferment xylose (5,6). Other yeasts suffer from similar limitations.

Strategies for engineering yeasts such as S. cerevisiae for the ability to ferment xylose are needed.

### SUMMARY OF THE INVENTION

One aspect of the invention is directed to recombinant yeasts comprising one or more recombinant genes. In some versions, the one or more recombinant genes are configured to express one or more enzymes.

In some versions, the one or more enzymes comprise any one or more of: a xylose reductase comprising a sequence at least 90%, at least 95%, or at least 99% identical to SEQ ID NO:1; a xylitol dehydrogenase comprising a sequence at least 90%, at least 95%, or at least 99% identical to SEQ ID NO:3; and a xylulokinase comprising a sequence at least 90%, at least 95%, or at least 99% identical to SEQ ID NO:5. In some versions, the one or more recombinant genes are configured to express the xylose reductase. In some versions, the one or more recombinant genes are configured to express the xylitol dehydrogenase. In some versions, the one or more recombinant genes are configured to express the xylulokinase.

In some versions, the recombinant yeast is of a species unable to consume xylose in native form and wherein the recombinant yeast is capable of consuming xylose. In some versions, the recombinant yeast is from the genus Saccharomyces. In some versions, the recombinant yeast is Saccharomyces cerevisiae.

In some versions, the recombinant yeast is capable of consuming xylose under anaerobic conditions. In some versions, the recombinant yeast is capable of consuming xylose in a culture medium consisting of 1% w/v yeast extract, 2% w/v peptone, 2% w/v xylose, and water under anaerobic conditions. In some versions, the recombinant yeast is capable of producing ethanol in a culture medium consisting of 1% w/v yeast extract, 2% w/v peptone, 2% w/v xylose, and water under anaerobic conditions. In some versions, the recombinant yeast is capable of growing, consuming xylose, and/or producing ethanol in a culture medium consisting of 1% w/v yeast extract, 2% w/v peptone, 2% w/v xylose, and water under anaerobic conditions.

In some versions, the recombinant yeast exhibits enhanced growth, enhanced xylose consumption, and/or enhanced ethanol production in a culture medium consisting of 1% w/v yeast extract, 2% w/v peptone, 2% w/v xylose, and water under anaerobic conditions with respect to a control yeast lacking the xylose reductase, the xylitol dehydrogenase, and the xylulokinase and comprising: a control xylose reductase comprising a sequence of SEQ ID NO:7; a control xylitol dehydrogenase comprising a sequence of SEQ ID NO:8; and a control xylulokinase comprising a sequence of SEQ ID NO:9.

Another aspect of the invention is directed to methods of consuming xylose. Some versions comprise culturing a recombinant yeast of the invention in a culture medium comprising xylose over a time period, wherein the recombinant yeast consumes at least some of the xylose over the time period.

In some versions, at least 50% by mass of the xylose initially present at a beginning of the time period is consumed over the time period.

In some versions, the recombinant yeast produces ethanol over the time period. In some versions, at least 1 g of ethanol per liter of the culture medium is produced over the time period.

In some versions, the culture medium includes no more than 400 μM, 100 μM, 25 μM, 5 μM, 2.5 μM, or 1 μM of dissolved oxygen over the time period.

In some versions, the time period is from 1 to 150 hours.

In some versions, the medium comprises biomass. In some versions, the medium comprises lignocellulosic biomass.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A and 1B. Yeast species from the order Serinales generate different metabolic end-products based on oxygen levels. Production of ethanol, xylitol, and/or glycerol by species of Scheffersomyces and Spathaspora genera under moderate (shake flask—SF) and high (baffled flask—BF) aeration. Black squares: metabolite(s) mainly produced by each species based on yields. White squares: the species produced low yields or did not produce the metabolite. Complete data are in Tables 1A-1C.

FIGS. 2A and 2B. Scheffersomyces xylosifermentans and Spathaspora passalidarum ferment xylose into ethanol at high yield and titer. Consumption of xylose and production of biomass, ethanol, xylitol, and glycerol by Sc. xylosifermentans, Sp. passalidarum, Scheffersomyces amazonensis, and Scheffersomyces coipomoensis under moderate (shake flask—SF) and high (baffled flask—BF) aeration. Error bars indicate the standard deviation from the three biological replicates.

FIGS. 3A-3C. Species with multiple copies of XYL1 accumulate xylitol. Xylose fermentation by closely related species under moderate (shake flask—SF) and high aeration (baffled flask—BF). Error bars indicate the standard deviation from the three independent replicates.

FIGS. 4A-4C. Xylose reductase from Scheffersomyces xylosifermentans lacks a cofactor preference. FIGS. 4A and 4B. XR (FIG. 4A) and XDH (FIG. 4B) activities under moderate aeration and high aeration expressed in units (U) per mg protein [U (mg protein)⁻¹]. FIG. 4C. Production of ethanol, xylitol, and/or glycerol by the species tested for XR and XDH activities (reproduced from FIGS. 1A and 1B). Abbreviations: Spa—Spathaspora passalidarum, Sxy—Scheffersomyces xylosifermentans, Scoip—Scheffersomyces coipomoensis, Sama—Scheffersomyces amazonensis, and Scer—Saccharomyces cerevisiae (negative control). Error bars indicate the standard deviation from the three biological replicates. Asterisks denote significant differences between the activities on NADH and NADPH for each species (P<0.05).

FIGS. 5A-5C. Several genes related to xylose fermentation were upregulated only for ethanol producers at lower oxygenation. FIG. 5A. Xylose metabolism and related pathways. FIGS. 5B and 5C. Gene expression from RNA-Seq analysis. Dashed lines correspond to the <−1 and >1 confidence intervals for the log 2 fold change from RNA-Seq analysis (moderate aeration/high aeration). Asterisks indicate genes that did not exhibit differential expression. Positive and negative values indicate upregulated and downregulated genes, respectively. The organisms are presented in the following order from top to bottom for each gene: Sc. xylosifermentans, Sp. Passalidarum, Sc. coipomoensis, and Sc. amazonensis. Protein products related to each gene: HXK—hexokinase, PGII—phosphoglucose isomerase, PFK—phosphofructokinase, FBA1—fructose 1,6-bisphosphate aldolase, TPIH—triose phosphate isomerase, TDH—glyceraldehyde-3-phosphate dehydrogenase, PGK1—3-phosphoglycerate kinase, GPM1—phosphoglycerate mutase, ENO—enolase, PYK1—pyruvate kinase, PDC1—pyruvate decarboxylase, ADH1/2—alcohol dehydrogenase, ALD6—aldehyde dehydrogenase, GPD1—glycerol-3-phosphate dehydrogenase, GPP1—glycerol-3-phosphate phosphatase, ZWF1—glucose-6-phosphate dehydrogenase, SOL—6-phosphogluconolactonase, GND1—6-phosphogluconate dehydrogenase, RKI1—ribose-5-phosphate ketol-isomerase, RPE1—D-ribulose-5-phosphate 3-epimerase, TKL1—transketolase, TAL1—transaldolase, XYL1—xylose reductase, XYL2—xylitol dehydrogenase, XYL3—xylulokinase.

FIGS. 6A-6H. Metabolic and carbohydrate metabolic processes are highly affected by oxygenation. Enrichment Analysis for Sc. xylosifermentans, Sc. coipomoensis, Sc. amazonensis, and Sp. passalidarum. FIGS. 6A-6D. Biological processes enriched in the upregulated gene list (moderate aeration/high aeration). FIGS. 6E-6H. Biological processes enriched in the downregulated gene list (moderate aeration/high aeration).

FIG. 7A-7D. Saccharomyces cerevisiae with XYL genes from Scheffersomyces xylosifermentans ferments xylose anaerobically. FIG. 7A. Growth curves (OD) of Sc. xylosifermentans, Sc. coipomoensis, and Sc. cerevisiae strains carrying different XYL genes under anoxic conditions. FIG. 7B. Xylose consumption. FIG. 7C. Ethanol production. FIG. 7D. Xylitol accumulation. Error bars indicate the standard deviation from the three biological replicates. Asterisks denote significant differences related to S. cerevisiae+SstipitisXYL1/XYL2/XYL3). Abbreviations: SxylosiXYL1 and SxylosiXYL1/XYL2—XYL genes from Sc. xylosifermentans; ScoipXYL1 and ScoipXYL1/XYL2—XYL genes from Sc. coipomoensis; and SstipitisXYL1/XYL2/XYL3—XYL genes from Sc. stipites.

FIG. 8A-8D. Growth curves of Sc. stipites and S. cerevisiae with Sc. xylosifermentans XYL3 under anoxic conditions. Error bars indicate the standard deviation from the three biological replicates. Asterisks denote significant differences between S. cerevisiae+SstipitisXYL1/XYL2/XYL3. Letter “a” indicates significant differences between S. cerevisiae+SxylosiXYL1 and SxylosiXYL1/XYL2/XYL3; letter “b” corresponds to the significant differences between S. cerevisiae+SxylosiXYL1/XYL2 and SxylosiXYL1/XYL2/XYL3.

### DETAILED DESCRIPTION OF THE INVENTION

One aspect of the invention is directed to recombinant yeasts. The recombinant yeasts of the invention can be recombinant forms of any yeast. Exemplary yeasts include those from the genera Saccharomyces, Kluyveromyces, Candida, Pichia, Schizosaccharomyces, Hansenula, Kloeckera, Schwanniomyces, Arxula and Yarrowia, among others. Specific exemplary yeasts include S. cerevisiae, S. bulderi, S. barnetti, S. exiguus, S. uvarum, S. diastaticus, K. lactis, K. marxianus, K. fragilis, P. pastoris, P. methlanolica, and H. polymorpha among others. “Recombinant” used with respect to a yeast refers to a yeast that comprises a recombinant gene, a recombinant nucleic acid, and/or a recombinant polypeptide. A recombinant gene is a gene that comprises a recombinant nucleic acid sequence, is present within a cell in which it does not naturally occur, and/or is present in a different locus (e.g., genetic locus or on an extrachromosomal plasmid) within a particular cell than in the corresponding native cell. A recombinant nucleic acid or polypeptide is one comprising a sequence that is not naturally occurring.

The recombinant yeasts of the invention can comprise one or more recombinant genes. “Gene” as used herein refers to a nucleic acid sequence capable of producing a gene product and may include such genetic elements as a coding sequence together with any other genetic elements required for transcription and/or translation of the coding sequence. Such genetic elements may include a promoter, an enhancer, and/or a ribosome binding site (RBS), among others. In some versions, multiple genes are configured in an operon, in which multiple coding sequences are operationally connected to a single promoter. Each coding sequence and promoter pair in such instances are considered herein to constitute separate genes, despite comprising the same promoter. “Gene product” refers to product encoded and produced by a particular gene. Examples of gene products include mRNAs and polypeptide translated therefrom (such as the enzymes of the invention). In some versions, one or more of the recombinant genes of the invention can incorporated into a chromosome of the recombinant yeast. In some versions, of or more of the recombinant genes of the invention can be contained on an extra-chromosomal plasmid.

The recombinant genes of the invention can be configured to express one or more enzymes. “Expression” used with reference to a gene or a gene product, refers to the production of the gene product from the gene. The production of the gene product from the gene can occur directly, such as in the case with RNA, or indirectly, as in the case of a protein. Being configured to express one or more enzymes means that the gene comprises a sequence (e.g., a coding sequence and any additional genetic elements) suitable for expressing the one or more enzymes in a particular host, such as the recombinant yeasts of the invention.

The one or more enzymes of the invention can comprise any one or more of a xylose reductase, a xylitol dehydrogenase, and a xylulokinase.

Xylose reductases include enzymes characterized by Enzyme Commission (EC) number 1.1.1.307. Xylose reductases catalyze the conversion of xylose to xylitol. Exemplary xylose reductases of the invention comprise an amino acid sequence of SEQ ID NO:1 or an amino acid sequence at least at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO:1. The xylose reductase of SEQ ID NO:1 is a xylose reductase of Sc. xylosifermentans. The amino acid sequence of SEQ ID NO:1 is:

(SEQ ID NO: 1)
MVAPIPQIKLSSGHLMPSIGFGCWKLDKPTAGEQVYEAIKAGYRLEDGA
EDYGNEKEIGDGVKKAIDEGIVKREELFLVSKLWNNYHDPKNVETALDR
TLADLKTDYVDLFYIHFPIAFKFVPLEEKYPPAFYCGDGDNFHYEDVPI
LETWKALEKLVASGKIRSIGVSNFPGALLLDLLRGATIKPAVLQVEHHP
YLQQPRLIEFAQKAGLTVTAYSSFGPQSFVEMNQGRALKTPPLFENEVI
KAIAAKHNRAPAEVLLRWSAQRGIAVIPKSNLPERLVQNRGFNSFDLTK
EDFEEIAKLDINLRENDPWDWDHIPIFV

An exemplary coding sequence of SEQ ID NO:1 is SEQ ID NO:2:

(SEQ ID NO: 2)
ATGGTTGCCCCAATCCCACAAATCAAATTGAGCTCCGGTCACTTGATGC
CTTCCATCGGTTTCGGCTGTTGGAAGCTCGACAAGCCAACCGCCGGTGA
ACAAGTCTACGAAGCCATCAAGGCCGGTTACAGATTGTTCGACGGTGCT
GAGGACTATGGTAACGAAAAGGAAATCGGTGACGGTGTCAAGAAGGCTA
TTGATGAAGGTATCGTCAAGAGAGAAGAGCTCTTCCTTGTCTCCAAGTT
GTGGAACAACTACCACGACCCAAAGAACGTCGAGACTGCCTTGGACAGA
ACCCTCGCTGACCTTAAGACCGACTACGTTGACTTGTTCTATATCCACT
TCCCAATTGCCTTCAAGTTCGTCCCACTCGAGGAGAAGTACCCACCAGC
ATTCTACTGTGGTGACGGTGACAACTTCCACTACGAAGACGTCCCAATC
TTGGAGACCTGGAAGGCCCTCGAGAAGTTGGTCGCTTCCGGTAAGATTA
GATCCATCGGTGTCTCCAACTTCCCAGGTGCTTTGCTCTTGGACTTGCT
CAGAGGTGCTACCATCAAGCCAGCTGTTTTGCAAGTTGAGCACCACCCA
TACTTGCAACAACCAAGATTGATTGAGTTCGCCCAAAAGGCCGGTCTTA
CCGTCACTGCTTACTCTTCTTTCGGTCCTCAATCCTTCGTTGAGATGAA
CCAAGGTAGAGCTTTGAAAACCCCACCATTGTTCGAAAACGAGGTCATC
AAGGCTATTGCTGCCAAGCACAACAGGGCCCCAGCTGAGGTTTTGTTGA
GATGGTCTGCTCAAAGAGGTATTGCTGTCATTCCAAAGTCTAACCTCCC
AGAGAGATTGGTCCAAAACAGAGGTTTCAACAGCTTCGACTTGACCAAG
GAGGACTTCGAGGAGATCGCCAAGTTGGACATCAACTTGAGATTCAACG
ACCCATGGGACTGGGACCATATCCCAATCTTCGTTTAA

Xylitol dehydrogenases include enzymes falling under EC 1.1.1.9. Xylitol dehydrogenases catalyze the conversion of xylitol to xylulose. Exemplary xylitol dehydrogenases of the invention comprise an amino acid sequence SEQ ID NO:3 or an amino acid sequence at least at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO:3. The xylitol dehydrogenase of SEQ ID NO:3 is a xylitol dehydrogenase of Sc. xylosifermentans. The amino acid sequence of SEQ ID NO:3 is:

(SEQ ID NO: 3)
MTANPSLVLNKIDDITFETYDAPEITEPTDVLVEVKKTGICGSDIHYYA
HGKIGNFVLTKPMVLGHESSGVVTKIGKGVTSLKVGDKVAIEPGIPSRF
SDAYKSGHYNLCPHMCFAATPNSTEGEPNPPGTLCKYFKSPEDFLVKLP
EHVSLEMGALVEPLSVGVHASKLASVRFGDFVAVFGAGPVGLLAAAVAK
TFGAKGVIVIDIFDNKLQMAKDIGAATHIFNSKTGGDAAALIKAFDGHE
PTVVLECTGAEPCINQGVAILAQGGRFVQVGNAPAPVKFPITEFATKEL
TLFGSFRYGFNDYKTSVDIMDTNYQNGKEKAPIDFEQLITHRFKFDDAI
KAYDLVRAGSGAVKCIIDGPE

An exemplary coding sequence of SEQ ID NO:3 is SEQ ID NO:4:

(SEQ ID NO: 4)
ATGACTGCCAACCCATCCCTCGTTCTCAACAAGATCGACGACATCACCT
TCGAAACCTACGATGCCCCAGAGATCACGGAACCAACCGATGTTCTCGT
AGAAGTCAAGAAAACAGGTATCTGTGGTTCAGATATCCACTACTACGCC
CACGGTAAGATCGGTAACTTCGTGTTGACCAAGCCAATGGTTCTTGGCC
ACGAATCCTCTGGTGTCGTCACCAAGATCGGTAAGGGTGTTACCTCCCT
CAAGGTCGGAGACAAGGTCGCCATTGAGCCAGGTATTCCATCCAGATTC
AGTGACGCCTACAAGAGCGGTCACTACAACTTATGTCCACACATGTGCT
TCGCTGCCACTCCAAACTCCACCGAGGGCGAGCCAAACCCACCAGGAAC
CTTATGTAAGTACTTCAAGTCCCCAGAGGATTTCTTGGTCAAGTTGCCA
GAACACGTTTCCTTGGAAATGGGTGCTCTTGTCGAGCCATTGTCTGTCG
GTGTCCACGCTTCCAAGTTGGCTTCCGTCAGATTCGGTGATTTTGTCGC
TGTCTTCGGTGCTGGTCCAGTCGGTCTCTTAGCTGCTGCCGTCGCCAAG
ACCTTCGGTGCTAAGGGTGTCATTGTCATTGACATTTTCGACAACAAGT
TGCAAATGGCCAAGGACATCGGTGCTGCTACCCACATCTTCAACTCCAA
GACCGGCGGTGACGCCGCTGCCTTAATTAAGGCTTTCGACGGCCACGAG
CCAACCGTCGTTTTGGAATGTACTGGTGCTGAGCCATGTATCAACCAAG
GTGTCGCTATCTTGGCCCAAGGTGGTCGTTTCGTCCAAGTCGGTAACGC
CCCAGCCCCAGTGAAGTTCCCAATCACCGAGTTCGCTACCAAGGAACTC
ACCTTGTTCGGATCTTTCAGATACGGTTTCAACGATTACAAGACCTCTG
TCGACATCATGGACACCAACTACCAGAACGGTAAGGAAAAGGCCCCAAT
TGACTTCGAACAATTGATCACCCACAGATTCAAGTTCGACGACGCCATT
AAGGCCTACGACTTGGTCAGAGCTGGTAGTGGTGCTGTCAAGTGTATCA
TTGACGGTCCTGAGTAA

Xylulokinases include enzymes falling under EC 2.7.1.17. Xylulokinases catalyze the phosphorylation of xylulose to xylulose-5-phosphate. Exemplary xylulokinases of the invention comprise an amino acid sequence of SEQ ID NO:5 or an amino acid sequence at least at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO:5. The xylulokinase of SEQ ID NO:5 is a xylulokinase of Sc. xylosifermentans. The amino acid sequence of SEQ ID NO:5 is:

(SEQ ID NO: 5)
MTTSLYDSSELLFLGFDLSTQQLKIIVTNEKLAALKTYNVEFDVLNKDV
YGVHKGVIAIDNEEHKGAIVSPVFMWLDSLDQIFEDMKKDSFPENKVAG
VSGSCQQHGSVFWSKQAEAALAELQAKSTLTEQLKNAFTFKHSPNWQDH
STGKELAEFEKIIGADNLADISGSRAHYRETGLQIRKLATRFNPAKYNA
TWRISLVSSFLASVLLGKVTTIEEADACGMNLYDIKAGDFNEDLLAIAA
GVHPELDNVKKDSPEYVAGVTALREKLGKVRPISYESEGKISPYFVDRY
GFNPECQVYSFTGDNLATIISLPLAPNDALISLGTSTTVLIITKNYNPS
SQYHLFKHPTMPDHYMGMICYCNGSLAREKVRDEINEKYNVKDTKSWDK
FNEILDKSHSFNNKLGIYFPLGEIVPNAAAQTKRSILTAENKIVDVELG
EKNWVEDDDVSSIVESQTLSCRLRAGPMLSGSVKTTASTSPSPPPEGDD
KQHLHHVYKDLVDKFGELYTDGKKQTYESVTARPNRCYYVGGASNNGSI
IRKMGSILAPLNGNYKVEIPNACALGGAYKASWSYECEAKKEWIAYDKY
INRLFEKSDGMEKFEAEDKWLDYFTGVGLLAKMESELKHN

An exemplary coding sequence of SEQ ID NO:5 is SEQ ID NO:6:

(SEQ ID NO: 6)
ATGACCACCTCTCTCTACGACAGCTCCGAGCTGCTTTTCCTCGGGTTCG
ATCTCTCGACCCAGCAGTTGAAGATCATCGTCACCAACGAGAAGCTCGC
AGCCTTGAAGACCTACAACGTCGAGTTCGATGTGCTCAACAAGGACGTC
TATGGCGTCCACAAGGGTGTCATTGCTATTGACAACGAAGAACACAAGG
GTGCCATCGTTTCCCCAGTTTTCATGTGGTTGGACTCGCTTGACCAGAT
TTTCGAGGATATGAAGAAGGATTCGTTCCCTTTCAACAAGGTTGCTGGT
GTTAGTGGATCTTGTCAACAACACGGTTCTGTTTTCTGGTCCAAACAAG
CCGAAGCCGCTTTGGCCGAATTGCAAGCTAAGTCTACTTTGACCGAACA
GTTGAAAAACGCCTTCACCTTCAAGCACTCTCCAAACTGGCAGGACCAT
TCCACCGGTAAGGAGTTGGCTGAGTTCGAGAAGATCATCGGTGCCGATA
ACTTGGCCGATATCTCGGGTTCCAGAGCCCATTACCGTTTCACCGGTCT
TCAGATCAGAAAATTGGCTACCAGATTCAACCCAGCCAAGTATAATGCT
ACCTGGAGAATCTCATTGGTGTCTTCATTCTTGGCCAGTGTTTTGCTCG
GTAAAGTCACCACCATCGAAGAAGCAGATGCTTGTGGTATGAACCTTTA
CGATATCAAGGCTGGTGACTTCAACGAAGACCTCTTGGCCATCGCTGCC
GGTGTCCACCCAGAATTGGACAATGTCAAGAAAGACAGTCCAGAATACG
TCGCTGGTGTAACTGCTTTACGTGAAAAGTTGGGTAAGGTCCGTCCTAT
CTCTTACGAGAGTGAAGGTAAGATTTCTCCATACTTCGTGGACAGATAC
GGCTTCAACCCAGAGTGTCAGGTCTACTCTTTCACTGGAGACAACTTGG
CTACCATCATTTCGTTACCTCTTGCACCGAACGATGCCTTAATCTCGTT
AGGTACATCCACCACAGTGTTGATTATCACCAAAAACTACAACCCTTCG
TCTCAATACCACTTGTTCAAGCATCCAACAATGCCAGACCACTACATGG
GTATGATCTGCTACTGTAACGGTTCCTTGGCCAGAGAAAAGGTCAGAGA
CGAAATCAATGAGAAATACAACGTCAAGGACACCAAGTCTTGGGACAAG
TTCAACGAAATCTTGGACAAATCCCACTCTTTCAACAACAAGTTGGGTA
TCTACTTCCCATTGGGAGAGATTGTTCCTAATGCTGCTGCACAGACCAA
GAGATCTATTTTGACCGCCGAAAATAAGATTGTCGATGTCGAATTGGGC
GAAAAGAACTGGGTGGAGGATGATGATGTCTCCTCCATTGTTGAGTCCC
AGACATTGAGTTGTAGATTGAGAGCCGGCCCAATGTTGAGTGGAAGTGT
GAAGACAACTGCATCTACTTCTCCTTCTCCACCACCAGAAGGAGACGAC
AAGCAACACTTGCACCATGTCTACAAGGATTTGGTCGACAAATTCGGCG
AGTTGTACACCGATGGAAAGAAACAAACCTACGAGTCCGTCACTGCCAG
ACCTAACCGTTGTTACTACGTCGGTGGTGCTTCCAACAACGGAAGTATC
ATTCGTAAGATGGGTTCCATTTTGGCCCCTCTCAATGGTAACTACAAGG
TGGAGATTCCTAACGCCTGTGCCTTAGGTGGAGCCTACAAGGCCAGTTG
GAGTTACGAATGCGAGGCCAAGAAGGAATGGATCGCCTATGACAAGTAC
ATCAACCGTTTGTTTGAGAAGAGTGACGGAATGGAAAAGTTCGAGGCTG
AAGACAAGTGGCTTGACTACTTCACTGGAGTTGGCTTGTTAGCCAAGAT
GGAGAGCGAATTAAAGCACAACTAG

The terms “identical” or “percent identity”, in the context of two or more polypeptide or nucleic acid sequences, refer to two or more sequences or subsequences that are the same or have a specified percentage of amino acid residues or nucleotides that are the same, when compared and aligned for maximum correspondence, as measured using one of the sequence comparison algorithms described herein (or other algorithms available to persons of skill) or by visual inspection. For sequence comparison and identity determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is any nucleic acid or amino acid sequence described herein. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)). One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation I of 10, a cutoff of 100, M=5, N=−4, and a comparison of both strands. For amino acid sequences, the BLASTP program uses as defaults a wordlength (W) of 3, an expectation I of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in determining sequence identity of sequences described herein.

The recombinant genes of the invention can comprise a coding sequence of any one or more of the above-referenced enzymes operably linked to a promoter. In some versions, the promoter is heterologous to the coding sequence(s). The promoter can be a constitutive promoter or an inducible promoter. Non-limiting examples of suitable promoters for use within yeast cells are typically viral in origin and include the promoter of the mouse metallothionein I gene (Hamer et al. (1982) J. Mol. Appl. Gen. 1:273); the TK promoter of Herpes virus (McKnight (1982) Cell 31:355); the SV40 early promoter (Benoist et al. (1981) Nature (London) 290:304); the Rous sarcoma virus promoter; the cytomegalovirus promoter (Foecking et al. (1980) Gene 45:101); and the yeast ga14 gene promoter (Johnston et al. (1982) PNAS (USA) 79:6971; Silver et al. (1984) PNAS (USA) 81:5951.

The term “heterologous” refers to an element in an arrangement with another element that does not occur in nature. For example, a gene or protein that is heterologous to a given cell is a gene or protein that does not occur in the cell in nature. A promoter that is heterologous to a given coding sequence is a promoter that is not operably linked to the coding sequence in nature.

“Operationally connected” refers to a relationship between two genetic elements (e.g., a promoter and coding sequence), in which one of the genetic elements controls or affects the activity of the other genetic element. For example, a promoter is operably linked to a coding sequence if the promoter affects the transcription or expression of the coding sequence.

The recombinant genes of the invention can be codon-optimized for the particular microorganism in which they are introduced. Codon optimization can be performed for any nucleic acid by a number of programs, including “GENEGPS”-brand expression optimization algorithm by DNA 2.0 (Menlo Park, CA), “GENEOPTIMIZER”-brand gene optimization software by Life Technologies (Grand Island, NY), and “OPTIMUMGENE”-brand gene design system by GenScript (Piscataway, NJ). Other codon optimization programs or services are well known and commercially available.

In some versions of the invention, the one or more genes can be configured for overexpression. Overexpression is when a gene is caused to be transcribed at an elevated rate compared to the endogenous or basal transcription rate for that gene. In some versions, overexpression additionally includes an elevated rate of translation of the gene compared to the endogenous translation rate for that gene. Methods of testing for overexpression are well known in the art, for example transcribed RNA levels can be assessed using RT-PCR and protein levels can be assessed using SDS-PAGE gel analysis. The genes configured to express the enzymes of the invention can be overexpressed by placing the coding sequence under the control of a more active promoter, increasing the copy number of genes comprising the coding sequence, introducing a translational enhancer on a gene comprising the coding sequence, and/or modifying factors (e.g., transcription factors or genes therefor) that control expression of a gene comprising the coding sequence.

“Endogenous” used in reference to a genetic element means that the genetic element is native to the microorganism in which it is disposed.

“Exogenous” used in reference to a genetic element means that the genetic element is not native to the microorganism in which it is disposed.

An “isolated” biological component (such as a nucleic acid molecule, polypeptide, or cell) is a component that has been substantially separated or purified away from other biological components in which the component naturally occurs, such as other chromosomal and extrachromosomal DNA and RNA and proteins. Nucleic acid molecules and polypeptides that have been “isolated” include nucleic acid molecules and polypeptides purified by standard purification methods. The term also includes nucleic acid molecules and polypeptides prepared by recombinant expression in a cell as well as chemically synthesized nucleic acid molecules and polypeptides.

Exogenous nucleic acids can be introduced stably or transiently into a cell using techniques well known in the art, including electroporation, lithium acetate transformation, calcium phosphate precipitation, lipofection, infection, particle gun acceleration, DEAE-dextran mediated transfection, liposome-mediated transfection, conjugation, transduction, and the like.

The one or more genes of the invention can be introduced in a yeast in an expression vector. Suitable expression vectors include, but are not limited to viral vectors, phage vectors, bacteriophage vectors, plasmids, phagemids, cosmids, fosmids, bacterial artificial chromosomes, P1-based artificial chromosomes, yeast plasmids, yeast artificial chromosomes, and any other vectors specific for cells of interest.

Useful vectors can include one or more selectable marker genes to provide a phenotypic trait for selection of transformed cells. The selectable marker gene encodes a protein necessary for the survival or growth of transformed cells grown in a selective culture medium. Cells not transformed with the vector containing the selectable marker gene will not survive in the culture medium. Typical selection genes encode proteins that (a) confer resistance to antibiotics or other toxins, e.g., nourseothricin, neomycin, G418, hygromycin B, phleomycin, ampicillin, neomycin, methotrexate, chloramphenicol, kanamycin, or tetracycline, (b) complement auxotrophic deficiencies, or (c) supply critical nutrients not available from complex media.

Methods for transforming yeast cells with recombinant DNA and producing polypeptides therefrom are disclosed by Clontech Laboratories, Inc., Palo Alto, Calif., USA (in the product protocol for the “YEASTMAKER”-brand yeast transformation system kit); Reeves et al. (1992) FEMS Microbiology Letters 99:193-198; Manivasakam and Schiestl (1993) Nucleic Acids Research 21(18):4414-5; and Ganeva et al. (1994) FEMS Microbiology Letters 121:159-64. Expression and transformation vectors for transformation into many yeast strains are available. For example, expression vectors have been developed for the following yeasts: Candida albicans (Kurtz, et al. (1986) Mol. Cell. Biol. 6:142); Candida maltosa (Kunze et al. (1985) J. Basic Microbiol. 25:141); Hansenula polymorpha (Gleeson et al. (1986) J. Gen. Microbiol. 132:3459) and Roggenkamp et al. (1986) Mol. Gen. Genet. 202:302); Kluyveromyces fragilis (Das et al. (1984) J. Bacteriol. 158:1165); Kluyveromyces lactis (De Louvencourt et al. (1983) J. Bacteriol. 154:737) and Van den Berg et al. (1990) Bio/Technology 8:135); Pichia quillerimondii (Kunze et al. (1985) J. Basic Microbiol. 25:141); Pichia pastoris (Cregg et al. (1985) Mol. Cell. Biol. 5:3376; U.S. Pat. Nos. 4,837,148; and 4,929,555); Saccharomyces cerevisiae (Hinnen et al. (1978) Proc. Natl. Acad. Sci. USA 75:1929 and Ito et al. (1983) J. Bacteriol. 153:163); Schizosaccharomyces pombe (Beach et al. (1981) Nature 300:706); and Yarrowia lipolytica (Davidow et al. (1985) Curr. Genet. 10:380-471 and Gaillardin et al. (1985) Curr. Genet. 10:49). Genetic transformation systems for metabolic engineering have been developed specifically for a number of lipogenic yeasts including Mucor circinelloides (Zhang et al. (2007) Microbiology-Sgm 153, 2013-2025), Yarrowia lipolytica (Xuan et al. (1988) Current Genetics 14, 15-21), Rhodotorula glutinis (Li et al. (2012) Appl Microbiol Biotechnol 97(11):4927-36), Rhodosporidium toruloides (Zhu et al. (2012) Nature Communications, Vol. 3), Lipomyces starkeyi (Calvey et al. (2014) Current Genetics 60, 223-23; Oguro et al. (2015) Bioscience Biotechnology and Biochemistry 79, 512-515), and Trichosporon oleaginosus (Gorner et al. (2016) Green Chemistry 18, 2037-2046).

In some versions of the invention, the recombinant yeast is of a species unable to consume xylose in native form, and the introduction of the one or more enzymes of the invention into the yeast to generate a recombinant yeast of the invention confers the ability of the recombinant yeast to consume xylose. Among other species, species of Saccharomyces, such as S. cerevisiae, are unable to consume xylose in native form (Wohlbach et al. 2011 (18)) but are able to consume xylose when enzymes such as the xylose reductase, xylitol dehydrogenase, and xylulokinase are introduced therein. In some versions, the one or more enzymes confer the ability of the recombinant yeast to consume xylose under anaerobic conditions. Methods of generating anaerobic conditions for yeast culture are well known in the art.

In some versions, the recombinant yeast of the invention is capable of growing in a culture medium consisting of 1% w/v yeast extract, 2% w/v peptone, 2% w/v xylose, and water under anaerobic conditions. In some versions, the recombinant yeast of the invention is capable of growing in a culture medium consisting of 1% w/v yeast extract, 2% w/v peptone, 2% w/v xylose, and water under anaerobic conditions at a rate from 0.001 OD units/hour to 0.1 OD units/hour, such as from 0.002 OD units/hour to 0.05 OD units/hour, from 0.004 OD units/or to 0.025 OD units/hour, or about 0.01 OD units/hour. Growth of the recombinant yeast can be ascertained by detecting an increase in optical density over the course of the culture.

In some versions, the recombinant yeast of the invention is capable of consuming xylose in a culture medium consisting of 1% w/v yeast extract, 2% w/v peptone, 2% w/v xylose, and water under anaerobic conditions. In some versions, the recombinant yeast of the invention is capable of consuming xylose in a culture medium consisting of 1% w/v yeast extract, 2% w/v peptone, 2% w/v xylose, and water under anaerobic conditions at a rate from 0.005 g/L/h to 0.5 g/L/h, such as from 0.01 g/L/h to 0.25 g/L/h, from 0.02 g/L/h to 0.125 g/L/h, or about 0.05 g/L/h.

In some versions, the recombinant yeast of the invention is capable of producing ethanol in a culture medium consisting of 1% w/v yeast extract, 2% w/v peptone, 2% w/v xylose, and water under anaerobic conditions. In some versions, the recombinant yeast of the invention is capable of producing ethanol in a culture medium consisting of 1% w/v yeast extract, 2% w/v peptone, 2% w/v xylose, and water under anaerobic conditions at a rate from 0.002 g/L/h to 0.2 g/L/h, such as from 0.004 g/L/h to 0.1 g/L/h, from 0.008 g/L/h to 0.05 g/L/h, or about 0.02 g/L/h.

The xylose reductase, xylitol dehydrogenase, and/or xylulokinase enzymes of the invention can be effective to confer enhanced growth, xylose consumption, and enhanced ethanol production over the xylose reductase, xylitol dehydrogenase, and xylulokinase enzymes from other organisms, such as those from Scheffersomyces (Pichia) stipites. An exemplary xylose reductase from S. stipites has the amino acid sequence of SEQ ID NO:7:

SEQ ID NO: 7)
MPSIKLNSGYDMPAVGFGCWKVDVDTCSEQIYRAIKTGYRLFDGAEDYA
NEKLVGAGVKKAIDEGIVKREDLFLTSKLWNNYHHPDNVEKALNRTLSD
LQVDYVDLFLIHFPVTFKFVPLEEKYPPGFYCGKGDNFDYEDVPILETW
KALEKLVKAGKIRSIGVSNFPGALLLDLLRGATIKPSVLQVEHHPYLQQ
PRLIEFAQSRGIAVTAYSSFGPQSFVELNQGRALNTSPLFENETIKAIA
AKHGKSPAQVLLRWSSQRGIAIIPKSNTVPRLLENKDVNSFDLDEQDFA
DIAKLDINLRDNDPWDWDKIPIFV

An exemplary xylitol dehydrogenase from S. stipites has the amino acid sequence of SEQ ID NO:8:

(SEQ ID NO: 8)
MTANPSLVLNKIDDISFETYDAPEISEPTDVLVQVKKTGICGSDIHFYA
HGRIGNFVLTKPMVLGHESAGTVVQVGKGVTSLKVGDNVAIEPGIPSRF
SDEYKSGHYNLCPHMAFAATPNSKEGEPNPPGTLCKYFKSPEDFLVKLP
DHVSLELGALVEPLSVGVHASKLGSVAFGDYVAVFGAGPVGLLAAAVAK
TFGAKGVIVVDIFDNKLKMAKDIGAATHTFNSKTGGSEELIKAFGGNVP
NVVLECTGAEPCIKLGVDAIAPGGRFVQVGNAAGPVSFPITVFAMKELT
LFGSFRYGFNDYKTAVGIFDTNYQNGRENAPIDFEQLITHRYKFKDAIE
AYDLVRAGKGAVKCLIDGPE

An exemplary xylulokinase from S. stipites has the amino acid sequence of SEQ ID NO:9:

(SEQ ID NO: 9)
MTTTPFDAPDKLFLGFDLSTQQLKIIVTDENLAALKTYNVEFDSINSSV
QKGVIAINDEISKGAIISPVYMWLDALDHVFEDMKKDGFPFNKVVGISG
SCQQHGSVYWSRTAEKVLSELDAESSLSSQMRSAFTFKHAPNWQDHSTG
KELEEFERVIGADALADISGSRAHYRFTGLQIRKLSTRFKPEKYNRTAR
ISLVSSFVASVLLGRITSIEEADACGMNLYDIEKREFNEELLAIAAGVH
PELDGVEQDGEIYRAGINELKRKLGPVKPITYESEGDIASYFVTRYGFN
PDCKIYSFTGDNLATIISLPLAPNDALISLGTSTTVLIITKNYAPSSQY
HLFKHPTMPDHYMGMICYCNGSLAREKVRDEVNEKFNVEDKKSWDKFNE
ILDKSTDFNNKLGIYFPLGEIVPNAAAQIKRSVLNSKNEIVDVELGDKN
WQPEDDVSSIVESQTLSCRLRTGPMLSKSGDSSASSSASPQPEGDGTDL
HKVYQDLVKKFGDLYTDGKKQTFESLTARPNRCYYVGGASNNGSIIRKM
GSILAPVNGNYKVDIPNACALGGAYKASWSYECEAKKEWIGYDQYINRL
FEVSDEMNSFEVKDKWLEYANGVGMLAKMESELKH

In some versions, the recombinant yeasts of the invention exhibit enhanced growth, enhanced xylose consumption, and/or enhanced ethanol production in a culture medium with respect to a control yeast. “Control yeast” refers to a yeast of the same species and same genetic background as the recombinant yeasts of the invention, except for the xylose reductase, the xylitol dehydrogenase, and/or the xylulokinase as specified. In some versions, the control yeast lacks the xylose reductase, the xylitol dehydrogenase, and the xylulokinase of the invention and comprising the xylose reductase, the xylitol dehydrogenase, and the xylulokinase of S. stipites (e.g., SEQ ID NOS:7-9). In some versions, the recombinant yeasts of the invention exhibit enhanced growth, enhanced xylose consumption, and/or enhanced ethanol production at a rate at least 1.1-fold, at least 1.5-fold, at least 2-fold, at least 2.5-fold, at least 3-fold, at least 3.5-fold, at least 4-fold, at least 4.5-fold, or at least 5-fold of the respective rate of the control yeast. In some versions, the enhanced growth, the enhanced xylose consumption, and/or the enhanced ethanol production are exhibited in a culture medium consisting of 1% w/v yeast extract, 2% w/v peptone, 2% w/v xylose, and water under anaerobic conditions.

Some aspects of the invention are directed to methods of consuming xylose with the recombinant yeasts of the invention. The methods can comprise a recombinant yeast of the invention in a culture medium comprising xylose over a time period. The recombinant yeast can consume at least some of the xylose over the time period.

The recombinant yeasts of the invention are capable of consuming xylose in anaerobic and oxygen-limited conditions. Accordingly, in various versions, the culture medium includes no more than 600 μM, no more than 550 μM, no more than 500 μM, no more than 450 μM, no more than 400 μM, no more than 350 μM, no more than 300 μM, no more than 250 μM, no more than 200 μM, no more than 150 μM, no more than 100 μM, no more than 75 μM, no more than 50 μM, no more than 25 μM, no more than 20 μM, no more than 15 μM, no more than 10 μM, no more than 5 μM, no more than 2.5 μM, no more than 1 μM, no more than 0.5 μM, no more than 0.25 μM, no more than 0.1 μM, no more than 0.05 μM, no more than 0.01 μM, no more than 0.005 μM, or no more than 0.001 μM dissolved oxygen over the time period. Dissolved oxygen can be measured using any of a number of methods known in the art. One example is measuring dissolved oxygen with an InPro model 6950 probe (Mettler, Toledo, OH) (see, e.g., Aceituno F F, Orellana M, Torres J, Mendoza S, Slater A W, Melo F, Agosin E. Oxygen response of the wine yeast Saccharomyces cerevisiae EC1118 grown under carbon-sufficient, nitrogen-limited enological conditions. Appl Environ Microbiol. 2012 December; 78(23):8340-52).

In various versions, at least 1% by mass, at least 5% by mass, at least 10% by mass, at least 15% by mass, at least 20% by mass, at least 25% by mass, at least 30% by mass, at least 35% by mass, at least 40% by mass, at least 45% by mass, at least 50% by mass, at least 55% by mass, at least 60% by mass, at least 65% by mass, at least 70% by mass, at least 75% by mass, at least 80% by mass, at least 85% by mass, at least 90% by mass, or at least 95% by mass of the xylose initially present at a beginning of the time period is consumed over the time period. In some versions, up to 5% by mass, up to 10% by mass, up to 15% by mass, up to 20% by mass, up to 25% by mass, up to 30% by mass, up to 35% by mass, up to 40% by mass, up to 45% by mass, up to 50% by mass, up to 55% by mass, up to 60% by mass, up to 65% by mass, up to 70% by mass, up to 75% by mass, up to 80% by mass, up to 85% by mass, up to 90% by mass, up to 95% by mass, up to 99% by mass or more of the xylose initially present at a beginning of the time period is consumed over the time period.

In some versions of the invention, the recombinant yeast produces ethanol over the time period. In various versions, at least 0.5 g, at least 1 g, at least 1.5 g, at least 2 g, at least 2.5 g, at least 3 g, at least 3.5 g, at least 4 g, at least 4.5 g, at least 5 g of ethanol per liter of the culture medium is produced over the time period. In various versions, up to 1 g, up to 1.5 g, up to 2 g, up to 2.5 g, up to 3 g, up to 3.5 g, up to 4 g, up to 4.5 g, up to 5 g, up to 5.5 g, up to 6 g, up to 6.5 g, or more of ethanol per liter of the culture medium is produced over the time period.

In various versions of the invention, the time period is at least 1 hour, at least 2 hours, at least 3 hours, at least 4 hours, at least 5 hours, at least 10 hours, at least 15 hours, at least 20 hours, at least 25 hours, at least 30 hours, at least 35 hours, at least 40 hours, at least 45 hours, at least 50 hours, at least 55 hours, at least 60 hours, at least 65 hours, at least 70 hours, at least 75 hours, at least 80 hours, at least 85 hours, at least 90 hours, at least 95 hours, at least 100 hours, at least 110 hours, at least 120 hours, at least 130 hours, at least 140 hours, or at least 150 hours. In various versions of the invention, the time period is up to 1 hour, up to 2 hours, up to 3 hours, up to 4 hours, up to 5 hours, up to 10 hours, up to 15 hours, up to 20 hours, up to 25 hours, up to 30 hours, up to 35 hours, up to 40 hours, up to 45 hours, up to 50 hours, up to 55 hours, up to 60 hours, up to 65 hours, up to 70 hours, up to 75 hours, up to 80 hours, up to 85 hours, up to 90 hours, up to 95 hours, up to 100 hours, up to 110 hours, up to 120 hours, up to 130 hours, up to 140 hours, up to 150 hours, up to 175 hours, up to 200 hours or more.

In some versions of the invention, the medium comprises biomass. The biomass can be derived from any source, such as corn cobs, corn stover, cotton seed hairs, grasses, hardwood stems, leaves, newspaper, nut shells, paper, softwood stems, sorghum, switchgrass, waste papers from chemical pulps, wheat straw, wood, woody residues, mixed biomass species such as those produced by native prairie, and other sources.

In some versions, the medium can comprise lignocellulosic biomass.

In some versions, the medium can comprise pretreated lignocellulosic biomass. Methods of pretreating lignocellulosic biomass are well known in the art. See Kumar et al. 2017 (Kumar A K and Sharma S. Recent Updates on Different Methods of Pretreatment of Lignocellulosic Feedstocks: A Review. Bioresour. Bioprocess. (2017) 4:7); Kumar et al. 2009 (Kumar, P.; Barrett, D. M.; Delwiche, M. J.; Stroeve, P., Methods for Pretreatment of lignocellulosic Biomass for Efficient Hydrolysis and Biofuel Production. Industrial & Engineering Chemistry Research 2009, 48, (8), 3713-3729); Wang et al. 2013 (Wang H, Tucker M, Ji Y. Recent Development in Chemical Depolymerization of Lignin: A Review. (2013) Journal of Applied Chemistry. 2013:1-9), and Karlen et al. 2020 (Karlen S D, Fasahati P, Mazaheri M, Serate J, Smith R A, Sirobhushanam S, Chen M, Tymkhin V I, Cass C L, Liu S, Padmakshan D, Xie D, Zhang Y, McGee M A, Russell J D, Coon J J, Kaeppler H F, de Leon N, Maravelias C T, Runge T M, Kaeppler S M, Sedbrook J C, Ralph J. Assessing the viability of recovering hydroxycinnamic acids from lignocellulosic biorefinery alkaline pretreatment waste streams. ChemSusChem. 2020 Jan. 26). Examples include chipping, grinding, milling, steam pretreatment, ammonia fiber expansion (AFEX, also referred to as ammonia fiber explosion), ammonia recycle percolation (ARP), CO₂ explosion, steam explosion, ozonolysis, wet oxidation, acid hydrolysis, dilute-acid hydrolysis, alkaline hydrolysis, organosolv, ionic liquids, gamma-valerolactone, and pulsed electrical field treatment, among others.

In some versions of the invention, the medium can comprise depolymerized lignin, such as chemically depolymerized lignin. Methods of depolymerizing lignin are well known in the art. See Pandey et al. 2010 (Pandey M P, Kim C S. Lignin Depolymerization and Conversion: A Review of Thermochemical Methods. Chemical & Engineering Technology, 2010, Vol. 34, Issue 1, pp. 3-145) and Wang et al. 2013 (Wang H, Tucker M, Ji Y. Recent Development in Chemical Depolymerization of Lignin: A Review. Journal of Applied Chemistry, 2013, Volume 2013, Article ID 838645).

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

### Summary

Cost-effective production of biofuels from lignocellulose requires fermentation of D-xylose. Many yeast species within and closely related to the genera Spathaspora and Scheffersomyces natively assimilate and ferment xylose. Other species consume xylose inefficiently, leading to extracellular accumulation of xylitol. Xylitol excretion is thought to be due to the different cofactor requirements of the first two steps of xylose metabolism. Xylose reductase (XR) generally uses NADPH to reduce xylose to xylitol, while xylitol dehydrogenase (XDH) generally uses NAD⁺ to oxidize xylitol to xylulose, creating an imbalanced redox pathway. This imbalance is thought to be particularly consequential in hypoxic or anoxic environments.

We screened the growth of xylose-fermenting yeast species in high and low aeration and identified both efficient xylose fermenters (ethanol producers) and inefficient consumers (xylitol producers). Selected species were further characterized for their XR and XDH cofactor preferences by enzyme assays and gene expression patterns by RNA-Seq. Our data revealed that xylose metabolism is more redox balanced in some species, but it is strongly affected by oxygen levels. Under high aeration, most species switched from ethanol production to xylitol accumulation, despite the availability of ample oxygen to accept electrons from NADH. This switch was followed by decreases in enzyme activity and expression of genes related to xylose metabolism, suggesting bottlenecks in xylose fermentation are not always due to cofactor preferences. Finally, we expressed XYL genes from Scheffersomyces species in a strain of Saccharomyces cerevisiae. Recombinant S. cerevisiae expressing XYL1 from Scheffersomyces xylosifermentans, which encodes XR without a cofactor preference, showed improved anaerobic growth on xylose as the primary carbon source compared to S. cerevisiae strain expressing XYL genes from Sc. stipites.

This present work explored xylose-fermenting yeasts of the order Serinales in different aeration conditions. Collectively, our data do not support the hypothesis that xylitol accumulation occurs exclusively due to differences in cofactor preference between xylose reductase and xylitol dehydrogenase; instead, gene expression plays a major role in response to oxygen levels. We have also identified the yeast Sc. xylosifermentans as a source for genes that can be engineered into S. cerevisiae to improve xylose fermentation and biofuel production.

### Introduction

Xylose is the most abundant pentose comprised by hemicellulose in plants; therefore, robust microorganisms that can ferment this sugar are required for profitable biofuel production from lignocellulosic materials (1,2). The budding yeast Saccharomyces cerevisiae is widely used in biotechnological applications and is one of the most understood model microorganisms (3,4). However, S. cerevisiae lacks certain traits that limit its usefulness in lignocellulosic biofuel production, prompting some to investigate other yeast species as alternative biocatalysts. For example, S. cerevisiae does not have the ability to ferment xylose (5,6), while several non-conventional yeast species do this remarkably well (7).

Species belonging to the genera Spathaspora and Scheffersomyces (order Serinales under a recently proposed taxonomy (8), formerly the CUG-Ser1 major clade (9)) are known for their association with insects and their habitats, such as decomposing wood, and by their natural ability to assimilate and/or ferment xylose (10-12). Spathaspora passalidarum, Scheffersomyces stipites (syn. Pichia stipites), Scheffersomyces segobiensis, Scheffersomyces shehatae, Scheffersomyces coipomoensis, and Scheffersomyces ergatensis were the first members assigned to their respective clades (13,14). Sp. passalidarum and Sc. stipites have also been used as sources of genes that have been engineered in S. cerevisiae to confer the ability to ferment xylose (15-17). These non-conventional yeasts harbor three genes that encode enzymes for xylose metabolism: XYL1, which encodes xylose reductase (XR) for the reduction of xylose to xylitol; XYL2, encoding xylitol dehydrogenase (XDH) for the conversion of xylitol to xylulose; and XYL3, which encodes xylulokinase (XK) for the phosphorylation of xylulose to xylulose-5-phosphate (18). The genomes of most studied xylose-fermenting species contain a XR that preferentially utilizes NADPH as its primary cofactor and has lower affinity for NADH, while the XDH is strictly NAD⁺-dependent (19-21). A commonly articulated hypothesis is that different cofactor preferences lead to an imbalance during xylose catabolism; specifically, xylitol accumulates in anoxic environments or oxygen-limited conditions because little or no NAD⁺ can be regenerated without sufficient oxygen to act as a terminal electron acceptor (15,22).

Some yeast species have adopted genetic mechanisms that manage cofactor imbalances during xylose catabolism. Unlike Sc. stipites, Sp. passalidarum bears two copies of XYL1, which are named XYL1.1 and XYL1.2. The first one encodes an XR with NADPH affinity, while the enzyme encoded by the second copy prefers NADH over NADPH (15). Interestingly, xylose metabolism in this species is driven towards ethanol production, instead of xylitol accumulation, even in low aeration (23,24). Indeed, mutating the XR to prefer NADH over NADPH or inserting Sp. passalidarum XYL1.2 increases the ethanol productivity and alleviates cofactor imbalance in S. cerevisiae (15,25,26). In addition to enzyme specificity, expression of the XYL genes, particularly XYL2, in S. cerevisiae has been observed to impact the xylose utilization, as higher expression led to more efficient xylose fermentation (16,27). Although those factors seem to be important for xylose metabolism, genetically modified S. cerevisiae strains still cannot ferment xylose at rates comparable to glucose (5,28,29). The presence of a complete, integrated XYL pathway alone is not sufficient for xylose assimilation and/or fermentation by S. cerevisiae (27,30), suggesting that other genetic and regulatory mechanisms are required for efficient xylose metabolism. For this reason, investigations of native xylose-fermenting yeast species may provide novel strategies to overcome this problem in S. cerevisiae, which has impeded bioenergy research for decades.

The overall aims of this work are to understand how some species belonging to the genera Spathaspora and Scheffersomyces can efficiently ferment xylose into ethanol, why other species incompletely catabolize the pentose and accumulate xylitol, and how oxygenation can impact these outcomes. Although several members of these genera have been described in the taxonomic literature in recent years, little information is available for most of them, and most earlier studies have been confined to Sp. passalidarum and Sc. stipites. Our data show that xylose metabolism for species from the order Serinales is highly plastic and that oxygenation has a broad effect on the gene expression of xylolytic and interacting pathways. More critically, our data show that genes from Sc. xylosifermentans, such as xylose reductase, xylitol dehydrogenase, and/or xylulokinase genes, can be engineered at least into S. cerevisiae to improve xylose fermentation and biofuel production.

### Results

### Scheffersomyces and Spathaspora Switch Ethanol Production to Xylitol Accumulation or Glycerol Production During Respiration

Species belonging to the order Serinales, particularly members of the genera Scheffersomyces and Spathaspora (31), are known to harbor the uncommon ability to ferment xylose. We first determined the quantities of metabolites that these species produced under moderate oxygen-limited growth conditions with xylose as the primary carbon source (see Methods). Ethanol and xylitol were the major metabolites produced by Spathaspora and Scheffersomyces species during xylose fermentation, while glycerol was produced less frequently. We refer to ethanol and xylitol producers for species that primarily excreted these metabolites (based on yields) under moderate aeration in unbaffled shake flasks (SF). The titers, productivity rates, and yields related to the consumption of D-xylose and the production of biomass, ethanol, and xylitol under moderate and high aeration conditions are summarized in Tables 1A-1C.

The species that produced ethanol under oxygen-limited conditions (41.7% of the yeasts tested) were Scheffersomyces xylosifermentans, Sc. parashehatae, Sc. virginianus, Sc. shehatae, Sc. stipites, Sc. illinoinensis, Sc. cryptocercus, Spathaspora arborariae, Sp. passalidarum, and Sp. gorwiae. Remarkably, Sc. xylosifermentans, Sc. parashehatae, and Sp. passalidarum consumed all the xylose and reached maximum titers and yields of ethanol within 24 h of fermentation. After this time, the species respired the ethanol. The xylitol producers (45.8%) included Scheffersomyces coipomoensis, Sc. insectosa, Sc. amazonensis, Sc. quercinus, Sp. brasiliensis, Sp. suhii, Sp. roraimanensis, Sp. girioi, Sp. hagerdaliae, Sp. xylofermentans, and Candida (Spathaspora) materiae. Except for the production of ethanol by Sc. amazonensis, Sc. coipomoensis, and C. materiae, the yield and productivity of xylitol surpassed ethanol production, and these species accumulated the highest titers of xylitol compared to other xylitol producers. Out of the twenty-four species of Spathaspora and Scheffersomyces tested, three were not capable of fermenting xylose: Sc. spartinae and Sc. gosingicus showed minimal growth and modest consumption of sugar with no production of any metabolite examined, while Sc. ergatensis did not grow.

TABLE 1A
Production of ethanol and xylitol from D-xylose cultures
by Spathaspora and Scheffersomyces species.

| | | Xylose | | |
|---|---|---|---|---|
| Aeration | | consumption | Biomass | Y_{x/s} |
| condition | Yeast species | (%)* | (g L⁻¹) | (g g⁻¹)^{†} |
| Moderate | Sc. amazonensis | 99 | 9.90 ± 0.16 | 0.20 |
| (SF) | Sc. coipomoensis | 99 | 16.2 ± 0.51 | 0.18 |
| | Sc. cryptocercus | 99 | 9.01 ± 0.10 | 0.17 |
| | Sc. illinoinensis | 99 | 9.89 ± 0.43 | 0.19 |
| | Sc. insectosa | 99 | 13.1 ± 0.25 | 0.25 |
| | Sc. parashehatae | 99 | 10.1 ± 0.19 | 0.22 |
| | Sc. quercinus | 94 | 13.5 ± 0.05 | 0.29 |
| | Sc. shehatae | 99 | 10.2 ± 0.16 | 0.24 |
| | Sc. stipites | 99 | 10.8 ± 0.28 | 0.22 |
| | Sc. virginianus | 99 | 7.51 ± 0.11 | 0.13 |
| | Sc. xylosifermentans | 99 | 4.90 ± 0.31 | 0.08 |
| | Sp. arborariae | 99 | 10.4 ± 0.08 | 0.21 |
| | Sp. brasiliensis | 98 | 10.8 ± 0.30 | 0.21 |
| | Sp. girioi | 99 | 9.96 ± 0.10 | 0.19 |
| | Sp. gorwiae | 47 | 12.2 ± 0.22 | 0.50 |
| | Sp. hagerdaliae | 99 | 10.3 ± 0.50 | 0.20 |
| | Sp. materiae | 99 | 8.80 ± 0.20 | 0.17 |
| | Sp. passalidarum | 99 | 8.70 ± 0.23 | 0.15 |
| | Sp. roraimanensis | 100 | 7.50 ± 0.05 | 0.16 |
| | Sp. suhii | 98 | 9.40 ± 0.31 | 0.18 |
| | Sp. xylofermentans | 100 | 12.0 ± 0.20 | 0.23 |
| High | Sc. amazonensis | 99 | 19.2 ± 1.32 | 0.37 |
| (BF) | Sc. coipomoensis | 99 | 14.0 ± 0.72 | 0.27 |
| | Sc. cryptocercus | 99 | 22.7 ± 0.72 | 0.45 |
| | Sc. illinoinensis | 99 | 19.7 ± 1.45 | 0.38 |
| | Sc. insectosa | 99 | 15.1 ± 0.19 | 0.29 |
| | Sc. parashehatae | 99 | 15.6 ± 0.58 | 0.31 |
| | Sc. quercinus | 100 | 13.5 ± 0.12 | 0.27 |
| | Sc. shehatae | 100 | 13.6 ± 0.20 | 0.25 |
| | Sc. stipites | 100 | 14.4 ± 0.42 | 0.29 |
| | Sc. virginianus | 100 | 16.0 ± 0.70 | 0.32 |
| | Sc. xylosifermentans | 70 | 15.2 ± 0.91 | 0.43 |
| | Sp. arborariae | 92 | 19.8 ± 0.72 | 0.05 |
| | Sp. brasiliensis | 99 | 20.0 ± 0.89 | 0.38 |
| | Sp. girioi | 47 | 12.2 ± 0.13 | 0.49 |
| | Sp. gorwiae | 93 | 21.7 ± 0.35 | 0.46 |
| | Sp. hagerdaliae | 99 | 22.7 ± 1.12 | 0.44 |
| | Sp. materiae | 77 | 11.6 ± 0.25 | 0.29 |
| | Sp. passalidarum | 100 | 18.3 ± 1.08 | 0.36 |
| | Sp. roraimanensis | 99 | 15.4 ± 0.12 | 0.29 |
| | Sp. suhii | 100 | 15.5 ± 0.42 | 0.30 |
| | Sp. xylofermentans | 99 | 17.8 ± 0.15 | 0.34 |

*Xylose consumption (%)—initial D-xylose consumed.
^{†}Y_{x/s} (g g⁻¹)—biomass yield: correlation between biomass (ΔP) produced with sugar (ΔS) consumed.
SF—Shake-flask.
BF—Baffled flask.

TABLE 1B
Production of ethanol and xylitol from D-xylose cultures by
Spathaspora and Scheffersomyces species (continued).

| Aeration | | Ethanol | Y_{p/s}^{et} | Qp^{et} | η^{et} | Time |
|---|---|---|---|---|---|---|
| condition | Yeast species | (g L⁻¹) | (g g⁻¹)^{†} | (g/h L⁻¹)^{‡} | (%) [symbol] | (h) [symbol] |
| Moderate | Sc. amazonensis | 4.40 ± 0.17 | 0.09 | 0.09 | 18 | 48 |
| (SF) | Sc. coipomoensis | 4.77 ± 0.03 | 0.10 | 0.10 | 19 | 48 |
| | Sc. cryptocercus | 14.6 ± 0.42 | 0.29 | 0.30 | 57 | 48 |
| | Sc. illinoinensis | 14.5 ± 0.34 | 0.29 | 0.30 | 56 | 48 |
| | Sc. insectosa | 8.10 ± 0.13 | 0.16 | 0.11 | 31 | 72 |
| | Sc. parashehatae | 19.4 ± 0.20 | 0.39 | 0.81 | 78 | 24 |
| | Sc. quercinus | 4.00 ± 0.02 | 0.08 | 0.08 | 16 | 48 |
| | Sc. shehatae | 16.0 ± 0.15 | 0.32 | 0.33 | 63 | 48 |
| | Sc. stipites | 15.6 ± 0.31 | 0.31 | 0.33 | 61 | 48 |
| | Sc. virginianus | 19.1 ± 0.28 | 0.39 | 0.40 | 76 | 48 |
| | Sc. xylosifermentans | 21.9 ± 0.50 | 0.41 | 0.91 | 82 | 24 |
| | Sp. arborariae | 17.7 ± 0.20 | 0.36 | 0.37 | 70 | 48 |
| | Sp. brasiliensis | 5.66 ± 0.06 | 0.12 | 0.08 | 23 | 72 |
| | Sp. girioi | 5.27 ± 0.12 | 0.11 | 0.07 | 21 | 72 |
| | Sp. gorwiae | 1.21 ± 0.10 | 0.05 | 0.02 | 10 | 72 |
| | Sp. hagerdaliae | 8.60 ± 0.04 | 0.17 | 0.18 | 34 | 48 |
| | Sp. materiae | 3.54 ± 0.11 | 0.07 | 0.05 | 14 | 72 |
| | Sp. passalidarum | 18.9 ± 0.13 | 0.35 | 0.79 | 69 | 24 |
| | Sp. roraimanensis | 7.55 ± 0.01 | 0.17 | 0.16 | 34 | 48 |
| | Sp. suhii | 7.08 ± 0.20 | 0.14 | 0.10 | 28 | 72 |
| | Sp. xylofermentans | 4.22 ± 0.03 | 0.09 | 0.06 | 17 | 72 |
| High (BF) | Sc. amazonensis | 0.03 ± 0.00 | 0.00 | 0.00 | 0.1 | 72 |
| | Sc. coipomoensis | 1.28 ± 0.03 | 0.03 | 0.02 | 5 | 72 |
| | Sc. cryptocercus | 0.02 ± 0.00 | 0.00 | 0.00 | 0.1 | 72 |
| | Sc. illinoinensis | 0.03 ± 0.01 | 0.00 | 0.00 | 0.1 | 72 |
| | Sc. insectosa | 0.13 ± 0.08 | 0.00 | 0.00 | 0.5 | 72 |
| | Sc. parashehatae | 0.06 ± 0.01 | 0.00 | 0.00 | 0.2 | 72 |
| | Sc. quercinus | 0.08 ± 0.04 | 0.00 | 0.00 | 0.3 | 72 |
| | Sc. shehatae | 0.08 ± 0.01 | 0.00 | 0.00 | 0.3 | 72 |
| | Sc. stipites | 0.04 ± 0.00 | 0.00 | 0.00 | 0.2 | 72 |
| | Sc. virginianus | 0.12 ± 0.00 | 0.00 | 0.00 | 0.5 | 72 |
| | Sc. xylosifermentans | 0.12 ± 0.03 | 0.00 | 0.00 | 0.1 | 72 |
| | Sp. arborariae | 0.09 ± 0.01 | 0.00 | 0.00 | 0.4 | 72 |
| | Sp. brasiliensis | 0.05 ± 0.00 | 0.00 | 0.00 | 0.2 | 72 |
| | Sp. girioi | 0.47 ± 0.02 | 0.02 | 0.01 | 4 | 72 |
| | Sp. gorwiae | 0.02 ± 0.00 | 0.00 | 0.00 | 0.1 | 72 |
| | Sp. hagerdaliae | 0.08 ± 0.03 | 0.00 | 0.00 | 0.3 | 72 |
| | Sp. materiae | 0.02 ± 0.05 | 0.00 | 0.00 | 0.1 | 72 |
| | Sp. passalidarum | 0.12 ± 0.01 | 0.00 | 0.00 | 0.5 | 72 |
| | Sp. roraimanensis | 0.02 ± 0.00 | 0.00 | 0.00 | 0.1 | 72 |
| | Sp. suhii | 0.02 ± 0.01 | 0.00 | 0.00 | 0.1 | 72 |
| | Sp. xylofermentans | 0.02 ± 0.00 | 0.00 | 0.00 | 0.1 | 72 |

^{†}Y_{p/s}^{et} (g g⁻¹)—ethanol yield: correlation between ethanol (ΔP) produced with sugar (ΔS) consumed.
^{‡}Qp^{et}—ethanol productivity: ratio between ethanol concentration (g L⁻¹) and time (h). [symbol]
η^{et} (%)—conversion efficiency: percentage of the maximum theoretical ethanol yield (0.511 g ethanol per g D-xylose). [symbol]
Time by which maximum ethanol production (g L⁻¹) was reached by each species in shake flasks; by the end of fermentation for baffled flasks - when the species reached the maximum cell concentration.
SF—Shake-flask.
BF—Baffled flask.

TABLE 1C
Production of ethanol and xylitol from D-xylose cultures by
Spathaspora and Scheffersomyces species (continued).

| Aeration | | Xylitol | Y_{p/s}^{xyl} | Qp^{xyl} | η^{xyl} |
|---|---|---|---|---|---|
| condition | Yeast species | (g L⁻¹) | (g g⁻¹)^{†} | (g/h L⁻¹) [symbol] | (%) [symbol] |
| Moderate | Sc. amazonensis | 36.1 ± 0.37 | 0.73 | 0.75 | 80 |
| (SF) | Sc. coipomoensis | 32.1 ± 1.20 | 0.64 | 0.67 | 70 |
| | Sc. cryptocercus | 4.40 ± 0.04 | 0.09 | 0.09 | 10 |
| | Sc. illinoinensis | 1.77 ± 0.12 | 0.04 | 0.04 | 4 |
| | Sc. insectosa | 19.6 ± 0.89 | 0.38 | 0.27 | 61 |
| | Sc. parashehatae | 1.39 ± 0.12 | 0.03 | 0.06 | 3 |
| | Sc. quercinus | 25.4 ± 0.35 | 0.53 | 0.53 | 58 |
| | Sc. shehatae | 1.52 ± 0.10 | 0.03 | 0.03 | 3 |
| | Sc. stipites | 0.65 ± 0.02 | 0.01 | 0.01 | 1 |
| | Sc. virginianus | 1.61 ± 0.06 | 0.03 | 0.03 | 4 |
| | Sc. | 0.37 ± 0.00 | 0.01 | 0.02 | 1 |
| | xylosifermentans | | | | |
| | Sp. arborariae | 3.05 ± 0.29 | 0.06 | 0.06 | 7 |
| | Sp. brasiliensis | 24.9 ± 0.12 | 0.51 | 0.35 | 55 |
| | Sp. girioi | 22.5 ± 0.31 | 0.45 | 0.31 | 49 |
| | Sp. gorwiae | 0.00 ± 0.00 | 0.00 | 0.00 | 0 |
| | Sp. hagerdaliae | 16.2 ± 0.41 | 0.32 | 0.22 | 35 |
| | Sp. materiae | 33.5 ± 0.25 | 0.68 | 0.47 | 74 |
| | Sp. passalidarum | 0.85 ± 0.01 | 0.02 | 0.04 | 2 |
| | Sp. roraimanensis | 16.2 ± 0.22 | 0.38 | 0.34 | 41 |
| | Sp. suhii | 19.7 ± 0.17 | 0.40 | 0.27 | 44 |
| | Sp. | 25.0 ± 0.23 | 0.51 | 0.35 | 56 |
| | xylofermentans | | | | |
| High | Sc. amazonensis | 0.68 ± 0.00 | 0.01 | 0.01 | 2 |
| (BF) | Sc. coipomoensis | 0.30 ± 0.00 | 0.01 | 0.00 | 1 |
| | Sc. cryptocercus | 10.9 ± 0.08 | 0.22 | 0.15 | 24 |
| | Sc. illinoinensis | 0.87 ± 0.00 | 0.02 | 0.01 | 2 |
| | Sc. insectosa | 17.9 ± 0.14 | 0.36 | 0.25 | 39 |
| | Sc. parashehatae | 8.10 ± 0.20 | 0.16 | 0.11 | 18 |
| | Sc. quercinus | 1.99 ± 0.01 | 0.04 | 0.03 | 5 |
| | Sc. shehatae | 2.32 ± 0.02 | 0.05 | 0.03 | 5 |
| | Sc. stipites | 1.50 ± 0.05 | 0.03 | 0.02 | 3 |
| | Sc. virginianus | 3.27 ± 0.10 | 0.07 | 0.05 | 7 |
| | Sc. | 4.10 ± 0.29 | 0.12 | 0.06 | 13 |
| | xylosifermentans | | | | |
| | Sp. arborariae | 0.00 ± 0.00 | 0.00 | 0.00 | 0 |
| | Sp. brasiliensis | 0.00 ± 0.00 | 0.00 | 0.00 | 0 |
| | Sp. girioi | 0.00 ± 0.00 | 0.00 | 0.00 | 0 |
| | Sp. gorwiae | 3.51 ± 0.03 | 0.08 | 0.05 | 8 |
| | Sp. hagerdaliae | 1.49 ± 0.29 | 0.03 | 0.02 | 3 |
| | Sp. materiae | 4.43 ± 0.13 | 0.11 | 0.06 | 13 |
| | Sp. passalidarum | 0.05 ± 0.01 | 0.00 | 0.00 | 0.1 |
| | Sp. roraimanensis | 0.19 ± 0.08 | 0.00 | 0.00 | 0.4 |
| | Sp. suhii | 0.00 ± 0.00 | 0.00 | 0.00 | 0 |
| | Sp. | 0.66 ± 0.02 | 0.01 | 0.01 | 1 |
| | xylofermentans | | | | |

^{†}Y_{p/s}^{xyl} (g g⁻¹)—xylitol yield: correlation between xylitol (ΔP) produced with sugar (ΔS) consumed. [symbol]
Qp^{xyl} (g L⁻¹ h⁻¹)—xylitol productivity: ratio between xylitol concentration (g L⁻¹) and time (h). [symbol]
η^{xyl} (%)—conversion efficiency: percentage of the maximum theoretical xylitol yield (0.917 g xylitol per g D-xylose).
SF—Shake-flask.
BF—Baffled flask.

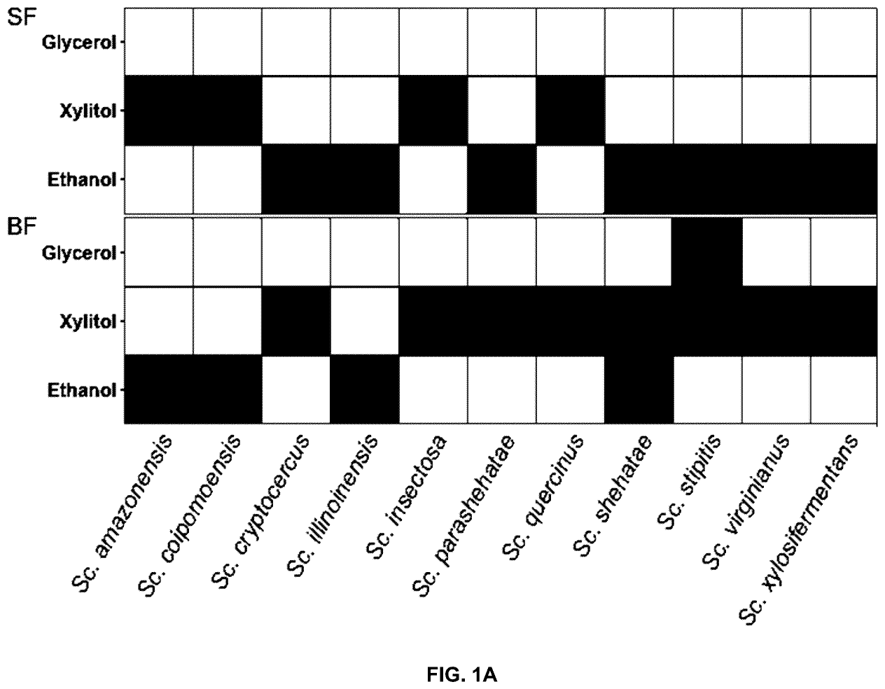

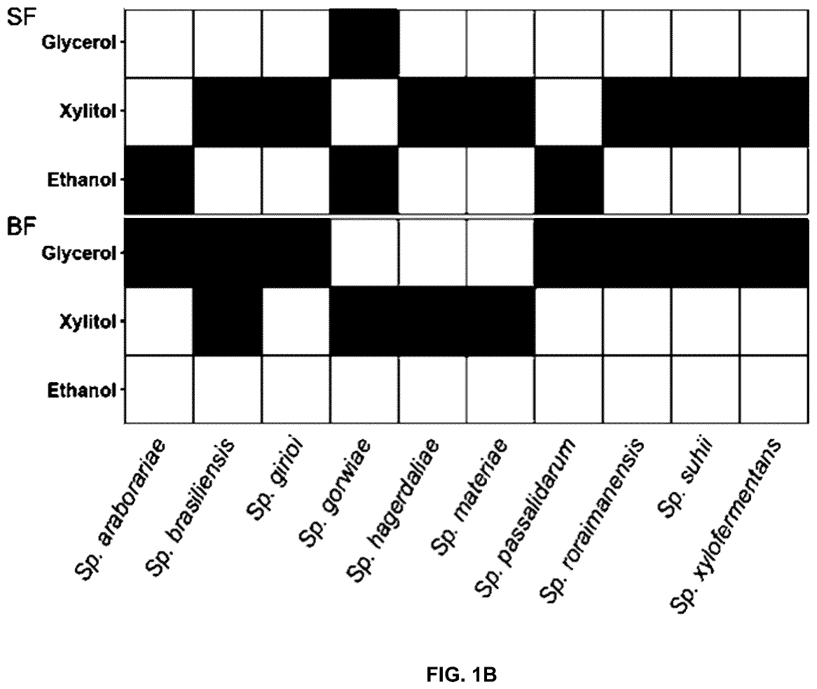

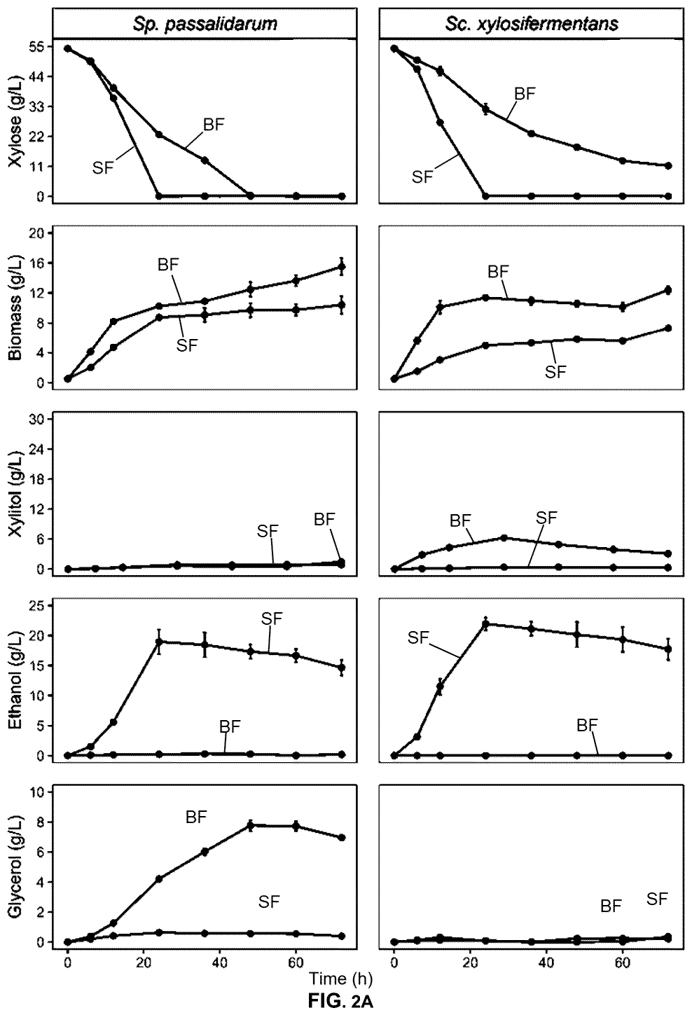

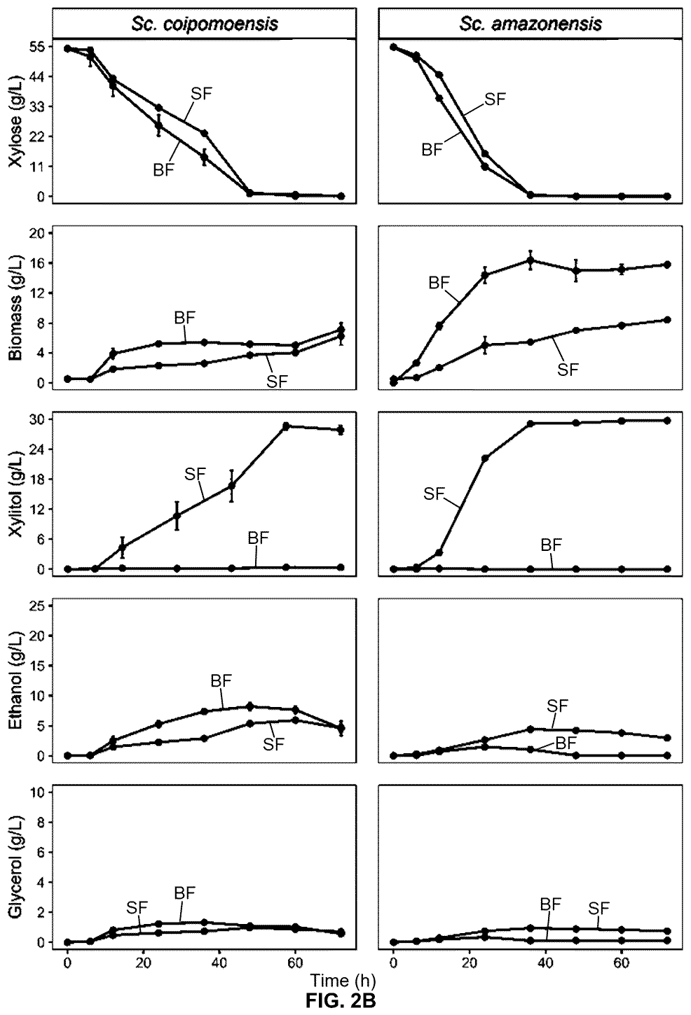

To verify the impact of the oxygenation on the xylose usage by Spathaspora and Scheffersomyces species, we also tested the species in baffled flasks (BF). This condition increases the volumetric mass transfer coefficient of oxygen (kLa), which is a parameter that determines the rate at which a gaseous compound can transfer between the gas and liquid phases (Li et al., 2013). Using an oxygen meter, we determined that the dissolved oxygen level in BF (4.74 mg L⁻¹) was nearly double the oxygen present in SF (2.63 mg L⁻¹). Although respiration dominated fermentation under high aeration, multiple species still fermented, while several species produced different primary metabolites in BF (FIGS. 1A and 1B). Sc. xylosifermentans, which presented high consumption of xylose and ethanol yields in SF, accumulated xylitol under high aeration and it did not consume xylose completely in 72 h. Sp. passalidarum produced glycerol, instead of ethanol, and the consumption of xylose was delayed in BF. For some species, especially xylitol producers, oxygen increased the rate of xylose utilization; xylose was consumed faster under high aeration than moderate oxygen-limited conditions. FIGS. 2A and 2B show the distinction between ethanol producers (Sp. passalidarum and Sc. xylosifermentans) and xylitol producers (Sc. coipomoensis and Sc. amazonensis). In contrast to the former two species, Sc. coipomoensis and Sc. amazonensis had slightly enhanced xylose consumption and produced ethanol, instead of xylitol, under high aeration.

For all the species studied here, the increased oxygen levels in BF favored the production of cell biomass. With greater oxygen availability in BF, the metabolism would be directed mainly towards cellular respiration. In general, the yeasts produced higher end-product (ethanol/xylitol) titers in SF compared to BF. Despite the xylitol production by the ethanol producers, this result suggests that most of the carbon was converted into carbon dioxide (CO₂) in BF.

Most Species with Multiple XYL1 Copies Accumulate Xylitol Under Moderate and High Aeration

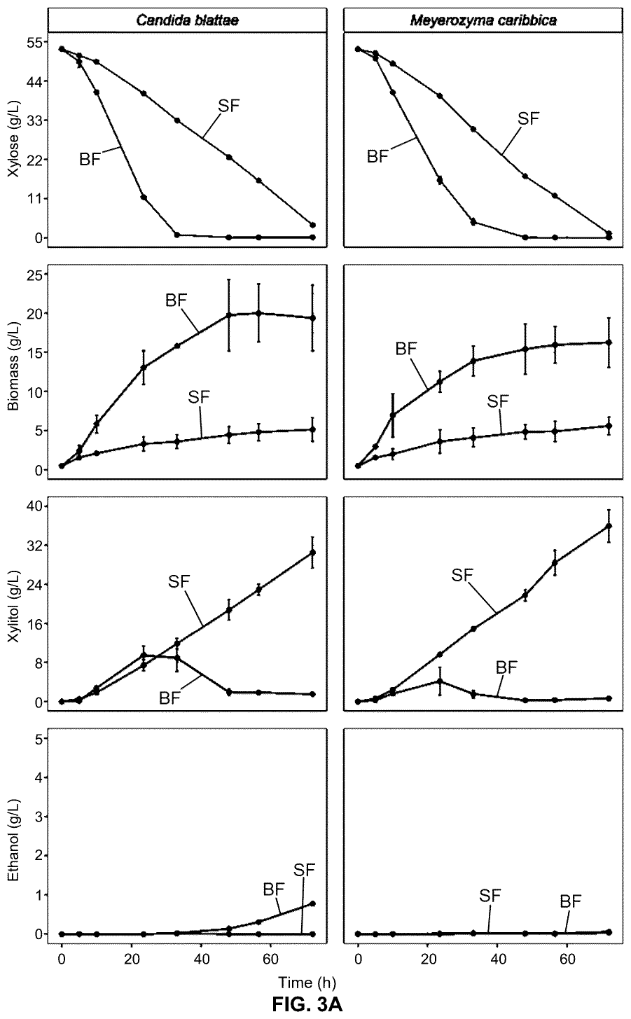

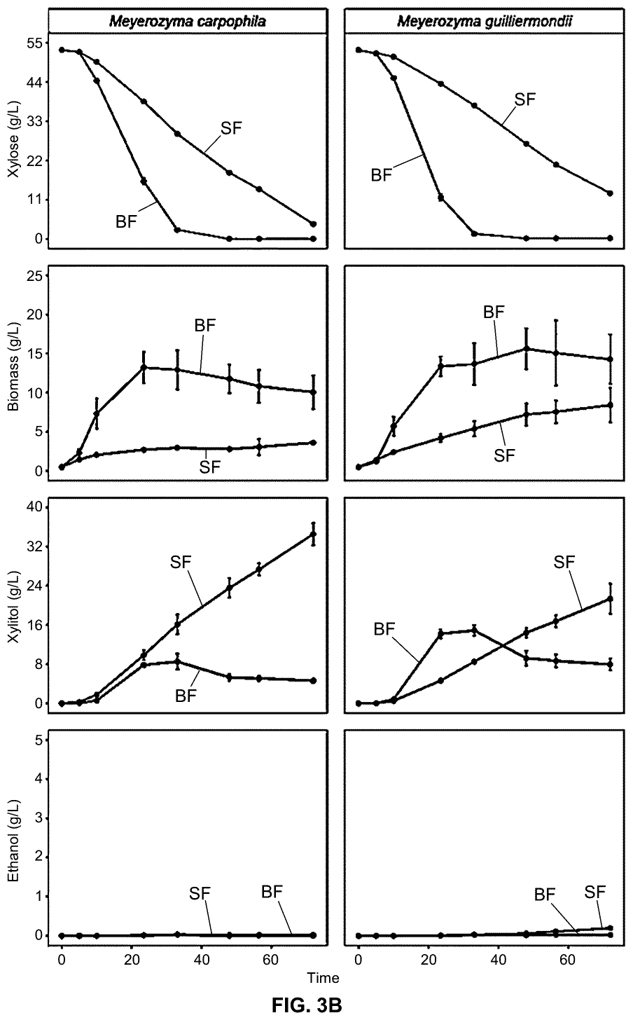

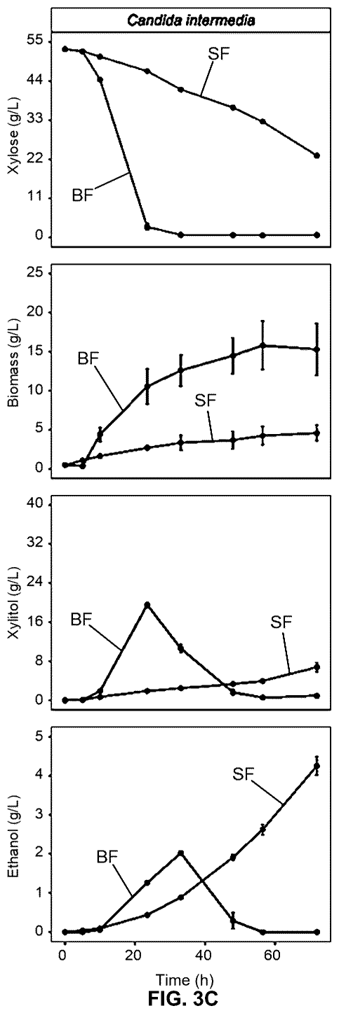

Recently, a study reported the presence of multiple copies of XYL1 in Candida intermedia, which harbors three copies of the gene, one of which (called XYL1.2) encodes a XR with higher affinity for NADH (19). Like the XYL1.2 from Sp. passalidarum, C. intermedia XYL1.2 displays dual cofactor specificity (15,19). A search for homologs across the order Serinales uncovered Candida blattae, Meyerozyma carpophila, Me. guilliermondii, and Me. caribbica as also having multiple XYL1 genes. To check if these species ferment xylose efficiently as Sp. passalidarum, and the influence of multiple copies of XYL1 genes on the xylose metabolism, we tested these species, as well as C. intermedia, during growth in YPX (1% yeast extract, 2% peptone, 5% xylose) under moderate and high aeration. Xylose was completely consumed by these strains under high aeration, while a significant amount of xylose remained unused under moderate conditions. These results suggested that xylose utilization by these closely related species was highly dependent on oxygen. All the species outside of the Scheffersomyces and Spathaspora clades accumulated xylitol in both conditions (FIGS. 3A-3C). Although the yield of xylitol surpassed the yield of ethanol produced during moderate aeration by C. intermedia, it was the only species able to achieve a significant titer of ethanol. Its fermentative capability may be due to the presence of an enzyme that favors the use of NADH. However, we do not know the cofactor preferences of the enzymes encoded by the copies of XYL1 from the other species.

Xylose Reductase from Scheffersomyces xylosifermentans has Dual Cofactor Affinity

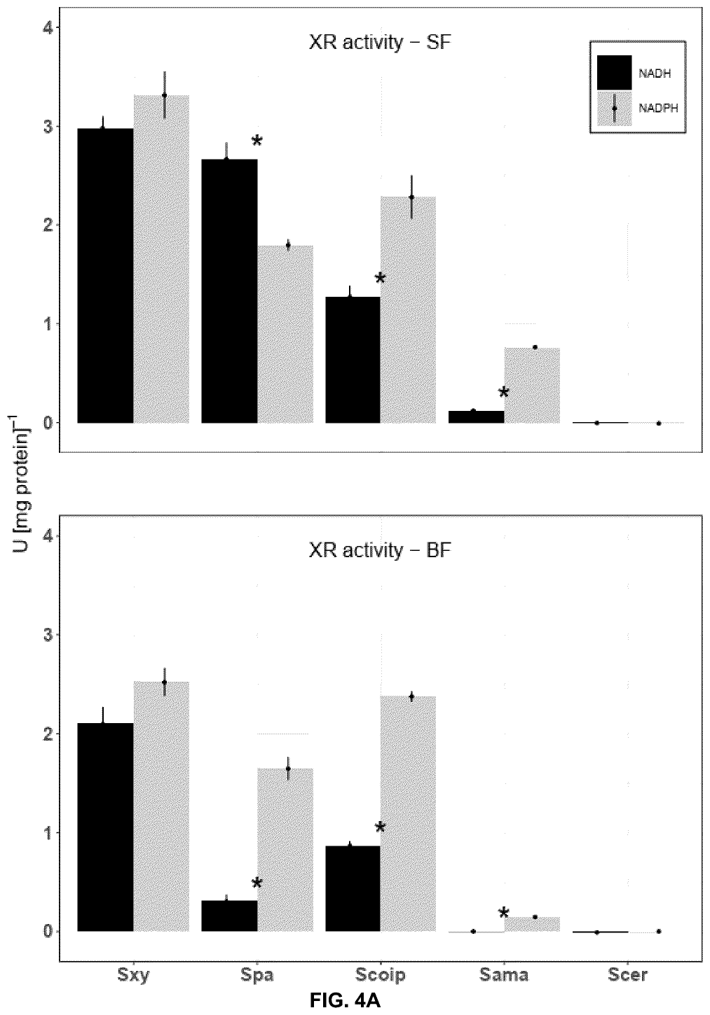

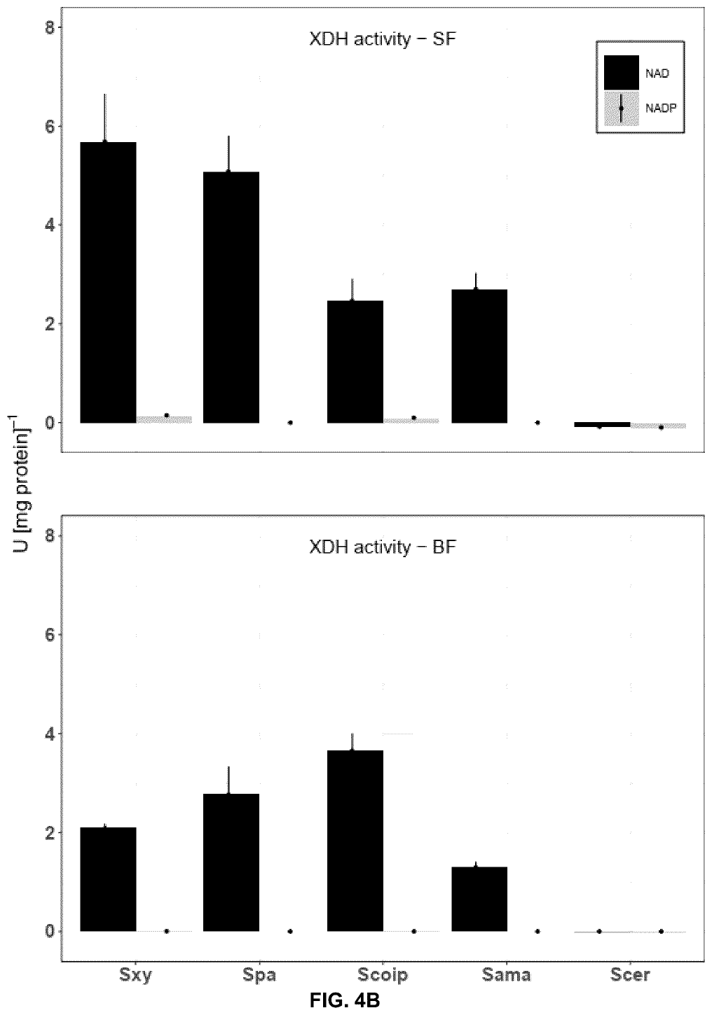

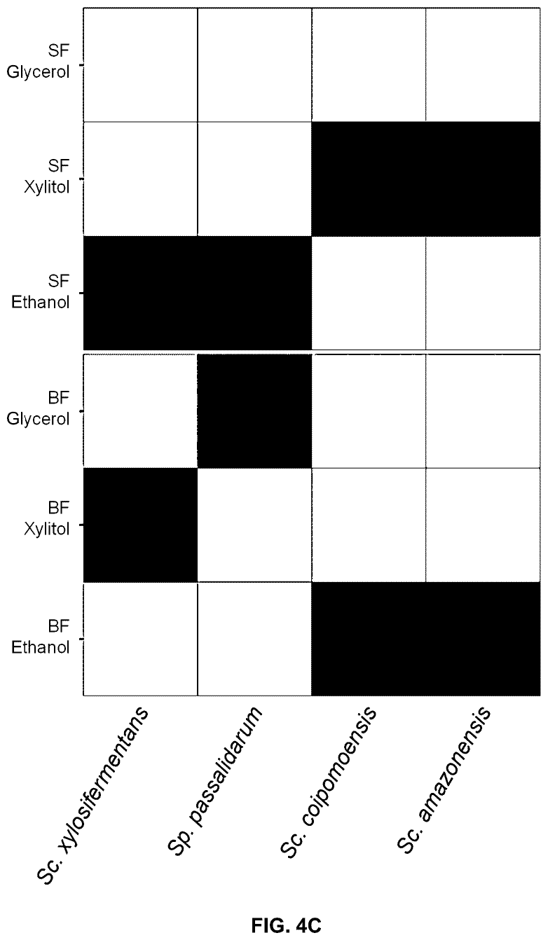

Xylitol accumulation during metabolic conversion of xylose has been proposed to result from imbalanced cofactor usage by XR and XDH. To determine the cofactor preference of the enzymes in the first steps of xylose metabolism, we measured the specific activity of XR and XDH from ethanol-producing Sp. passalidarum and Sc. xylosifermentans, as well as from xylitol-producing Sc. amazonensis and Sc. coipomoensis, in SF and BF (FIGS. 4A and 4B). The rationale for selecting Sc. xylosifermentans, Sc. coipomoensis, and Sc. amazonensis was that they harbor a single copy of the XYL1 gene, and they exhibited distinct phenotypes (ethanol versus xylitol production) under the tested conditions (FIG. 4C). In SF, XR of Sc. coipomoensis and Sc. amazonensis utilized NADH, but we observed greater XR activities with NADPH (P<0.05) (FIGS. 4A and 4B). On the other hand, there were no significant differences in Sc. xylosifermentans XR activities between the two cofactors. This result is interesting because, among these species, Sc. xylosifermentans showed the highest consumption of xylose in SF. In BF, the cofactor preference of the enzymes showed no significant differences, given to the presence of a single copy of XYL1.

Previous research showed that XR activities from Sp. passalidarum utilized both NADH and NADPH with higher activity for NADH under moderate oxygen-limited conditions and higher activity for NADPH in high aeration (15,23). Our results were consistent with those previous observations; Sp. passalidarum XR significantly preferred NADH over NADPH (P<0.05) in SF and significantly preferred NADPH (P<0.05) in BF. This switch in cofactor preference could have been enabled by the differential regulation of the two distinct XYL1 copies. This species also has two copies of XYL2 (32), but there is no data available in the literature showing the preference of XDH from each copy. Our results revealed that XDH was strictly NAD⁺-dependent not only for Sp. passalidarum, but for all four species tested. Low activities with NADP⁺ occurred for Sc. xylosifermentans and Sc. coipomoensis, but these were minimal compared to the activities with NAD⁺ (FIGS. 4A and 4B). For species that possess NADPH-preferring XRs, the exclusive utilization of NAD⁺ by XDH could potentially hinder the conversion of xylitol to xylulose under oxygen-limiting conditions, resulting in xylitol accumulation due to the previously proposed redox imbalance hypothesis (33,34). Nonetheless, the xylitol bottleneck observed in Sc. xylosifermentans with high aeration seems to require a different explanation because our enzyme assays suggested that its pathway was more redox-balanced due to its NADH-utilizing XR.

### Expression of Genes Related to Xylose Metabolism is Highly Affected by Oxygen

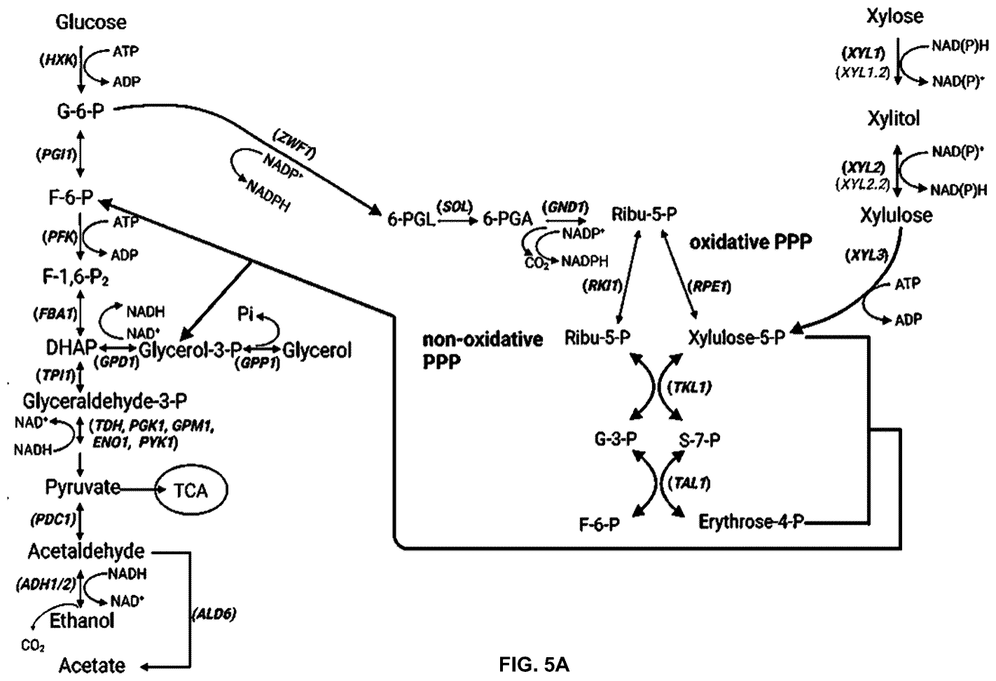

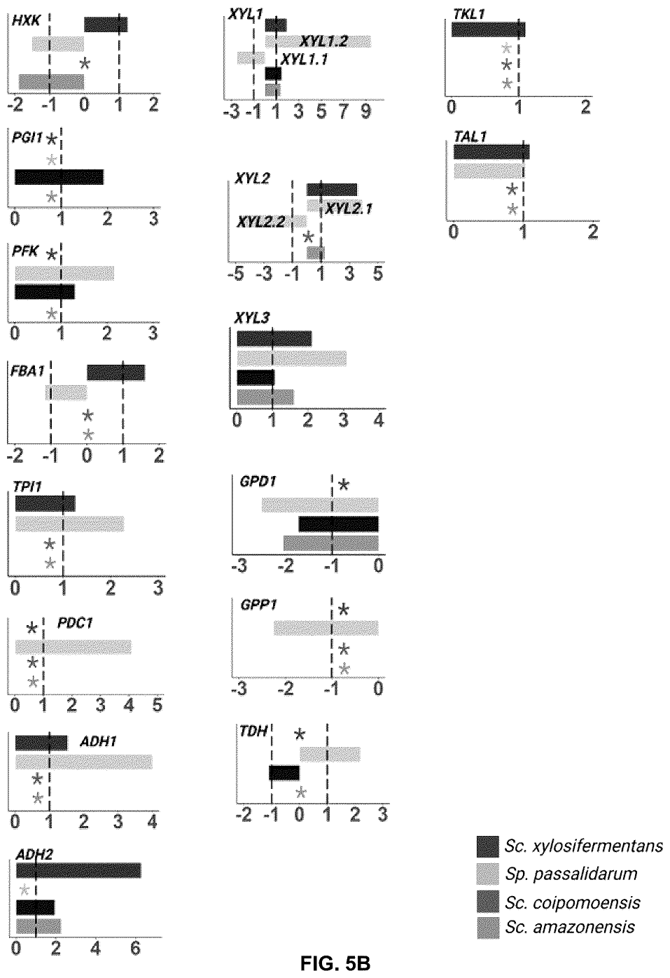

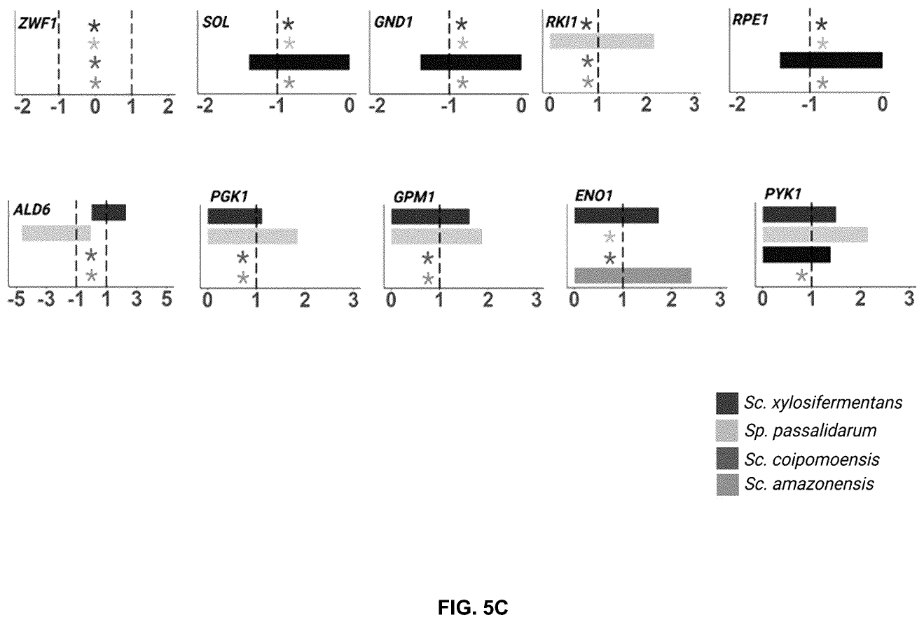

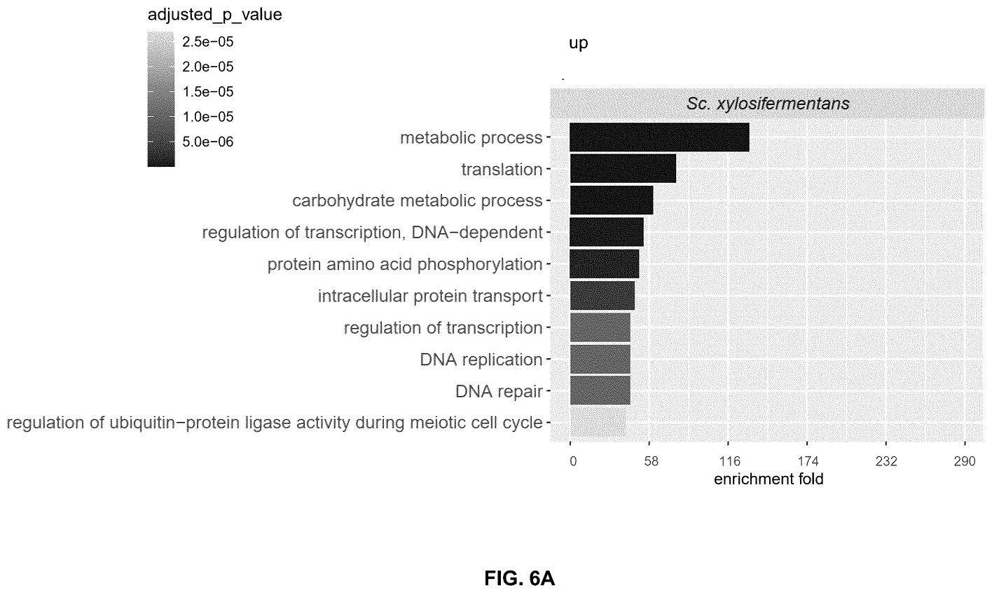

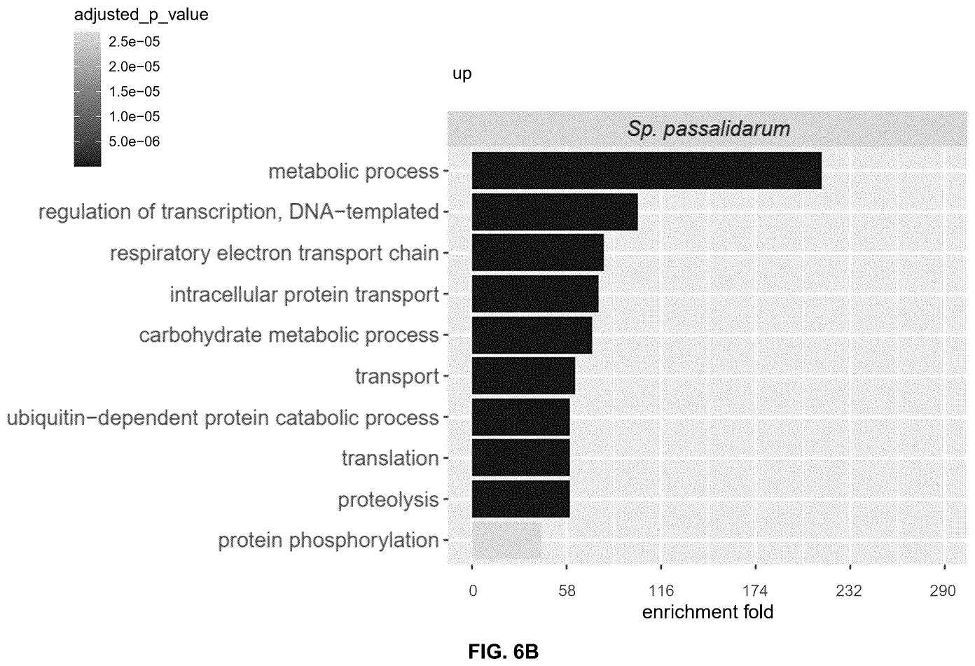

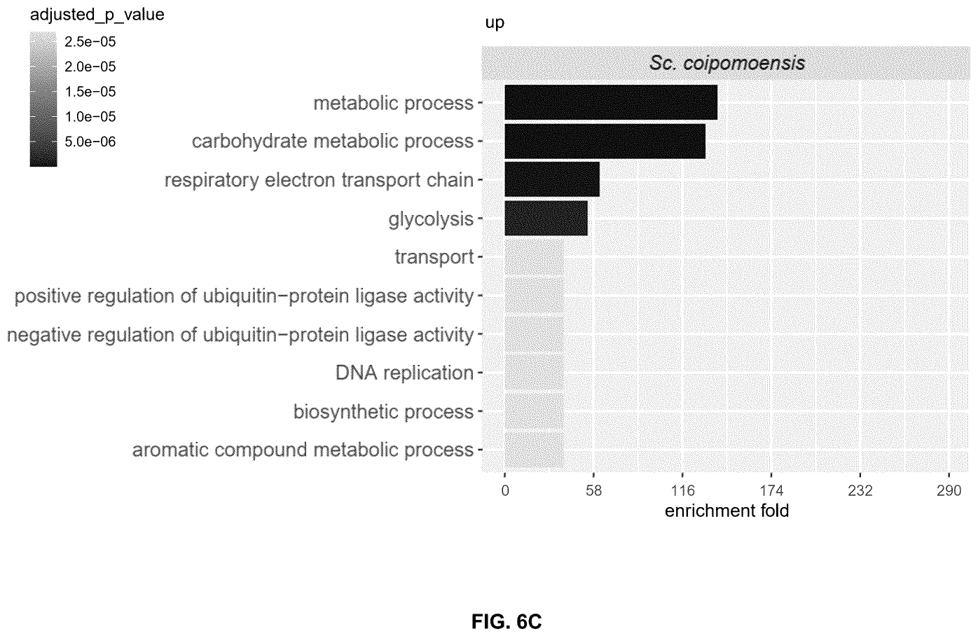

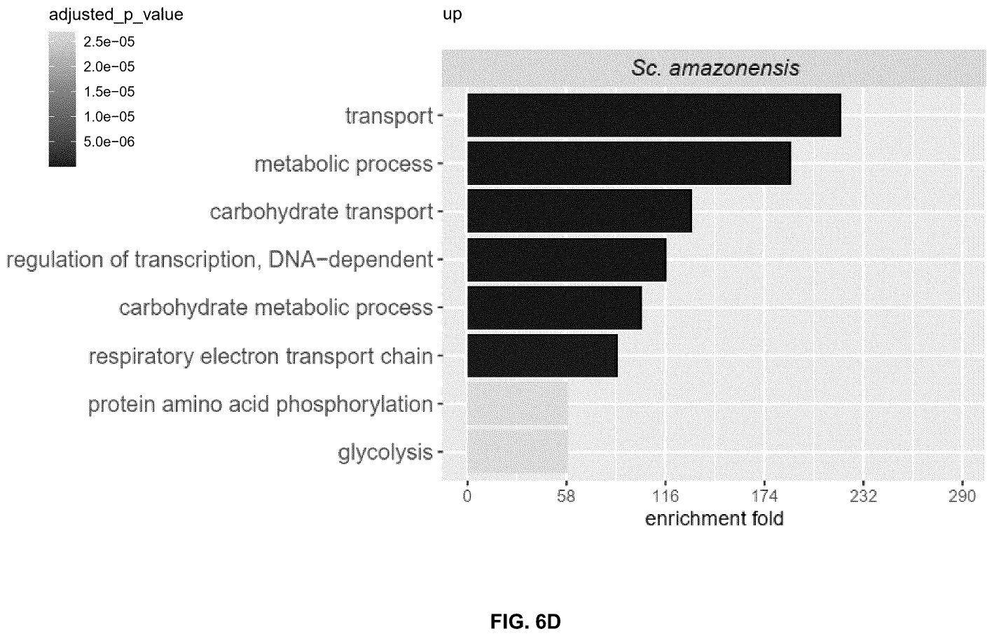

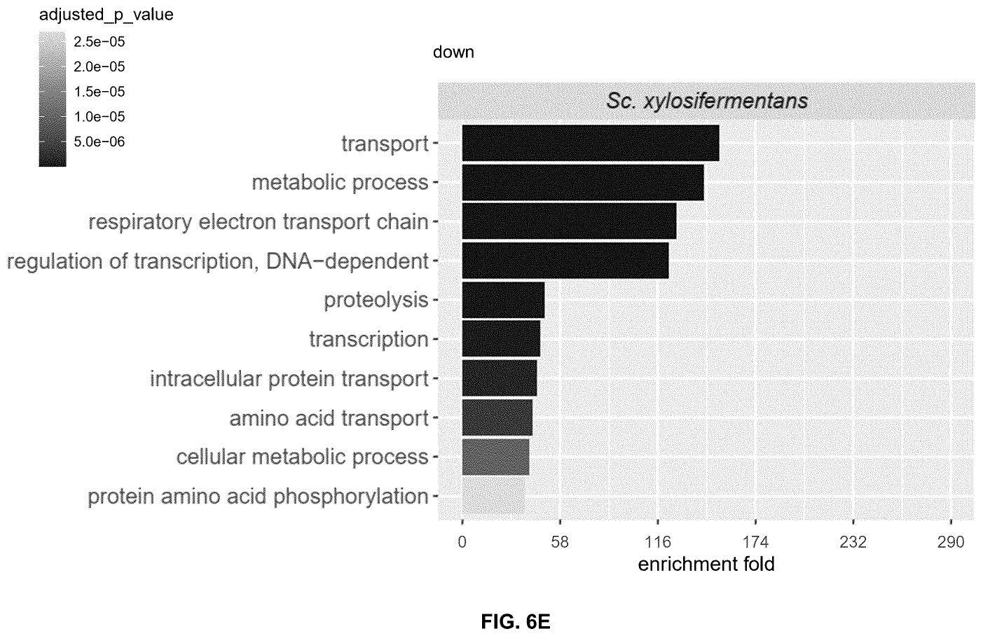

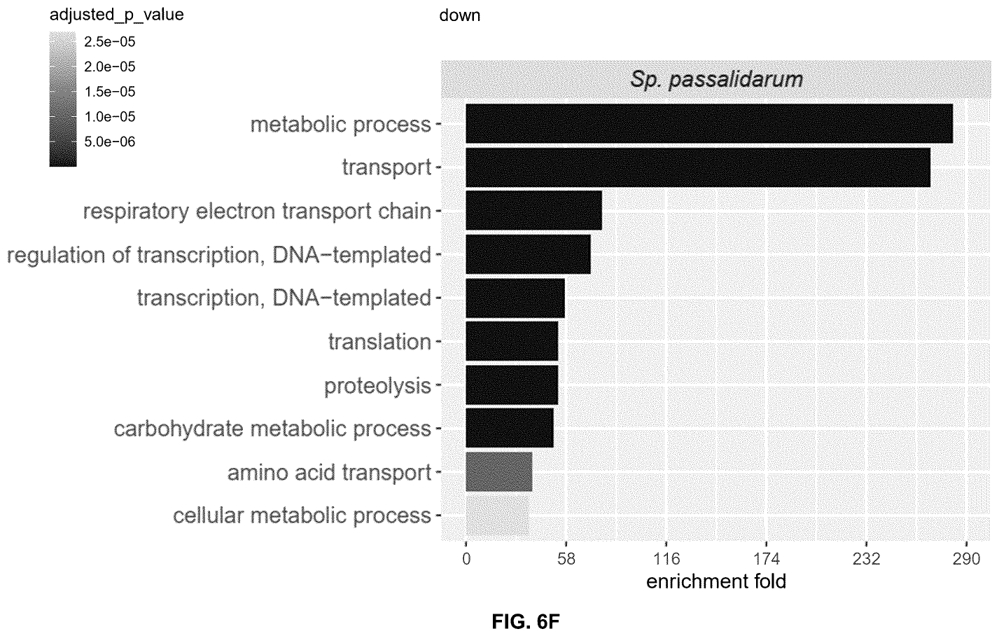

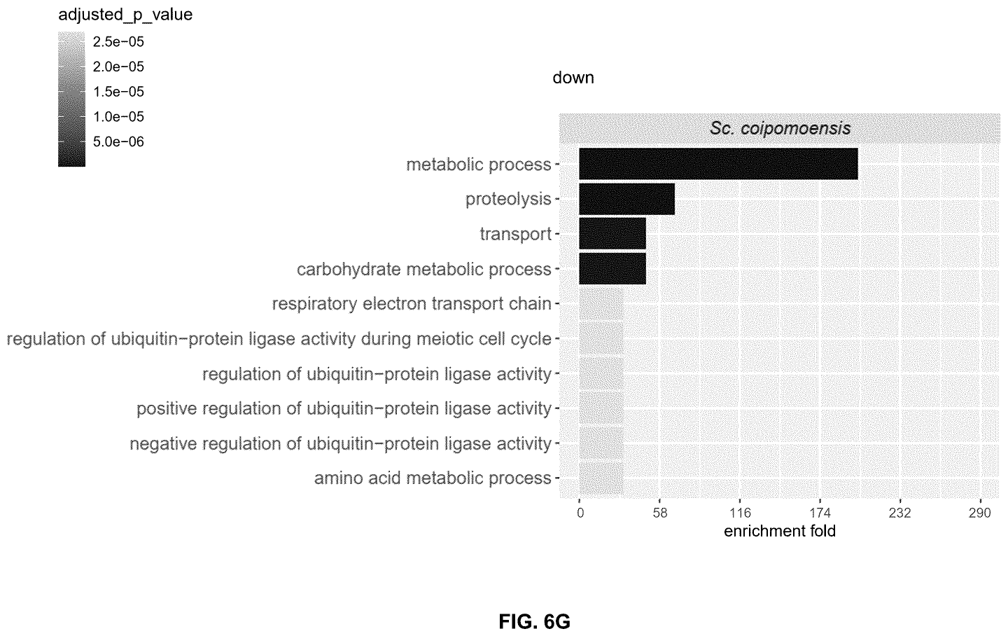

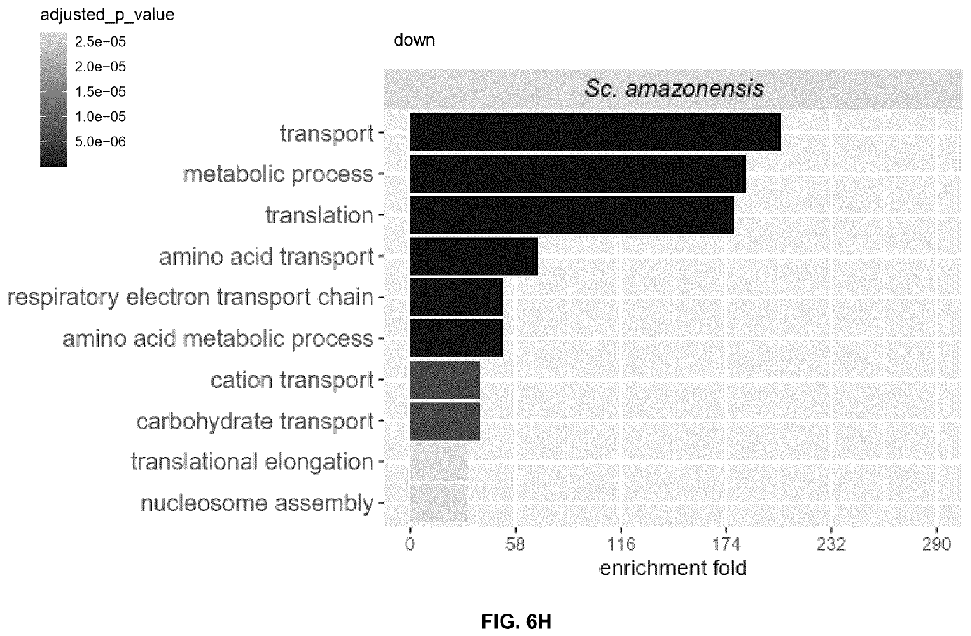

To determine whether oxygen levels (SF compared to BF) affected gene expression in a redox-independent way that could impact xylose-to-ethanol flux, we sequenced mRNA by taking samples in the mid-log phase from the same species used in the enzyme assays. After analyzing the data and filtering the results with specific thresholds (see Methods), we identified 2263, 2546, 2111, and 741 differential expressed genes (DEGs) for Sp. passalidarum, Sc. xylosifermentans, Sc. coipomoensis, and Sc. amazonensis, respectively. This differential expression analysis showed that xylose metabolism was highly affected by oxygenation. The genes encoding the first three steps of the xylose catabolism pathway were upregulated in SF (relative to BF) for all species except for Sc. coipomoensis XYL2, which was not differentially expressed. For Sp. passalidarum, XYL1.1 and XYL2.2 were downregulated in SF compared to BF, but XYL1.2 (the copy that encodes the XR with NADH affinity) and XYL2.1 were upregulated. Interestingly, XYL2 was among the 15 most upregulated genes in SF in Sc. xylosifermentans. FIGS. 5A-5C show the DEGs in key pathways from the comparison of SF and BF. Genes related to the non-oxidative phase of the pentose phosphate pathway (PPP), such as TKL1 and TAL1, were also upregulated in SF in ethanol producers under moderate aeration, but there were no DEGs for xylitol producers related to those genes. From the oxidative portion of the PPP that is not essential for xylose catabolism, some genes, such as SOL1, GND1, and RPE1, were downregulated in Sc. coipomoensis. Outside of the PPP, genes that include TPIH, PDC1, PGK1, GPM1 and ADH1 were also upregulated in SF only in ethanol producers. ADH1 and ADH2 are two genes related to ethanol production in Sc. stipites (35). While ADH1 was differentially expressed only in ethanol producers, ADH2 was also upregulated in xylitol producers and was the highest DEG in Sc. xylosifermentans. Although the ethanol producers presented the upregulation of key genes related to xylose metabolism in SF, several genes that were up- or down-regulated in the four species were enriched in biological processes, such as metabolic and carbohydrate metabolic processes (FIGS. 6A-6H). These biological processes, along with electron transport, likely have important roles that affect the observed phenotypes, suggesting that relevant pathways and genes related to metabolism were highly affected in the conditions tested.

XYL1 from Scheffersomyces xylosifermentans Enhanced Xylose Fermentation by Saccharomyces cerevisiae in Anoxic Conditions

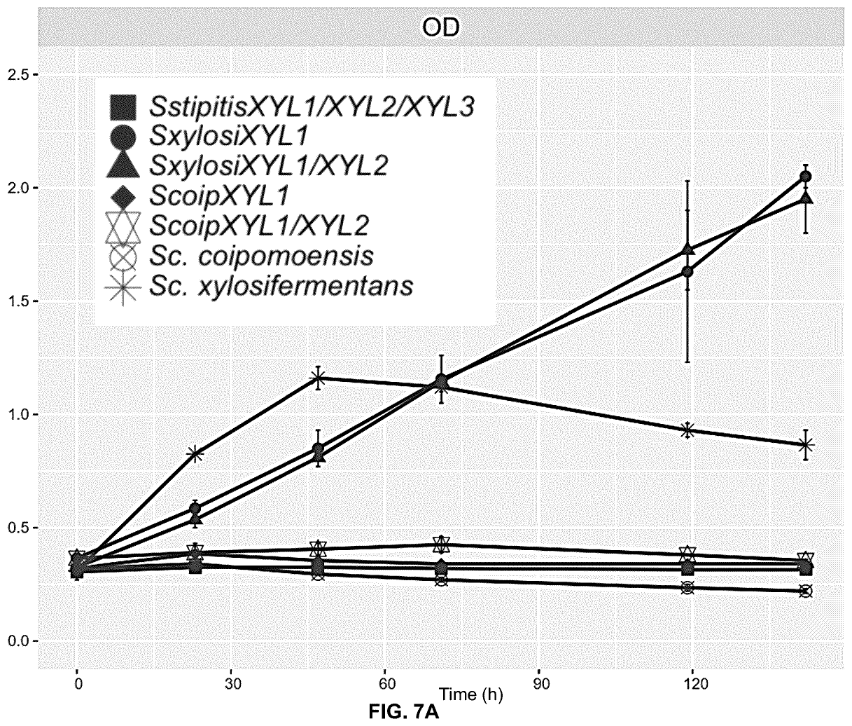

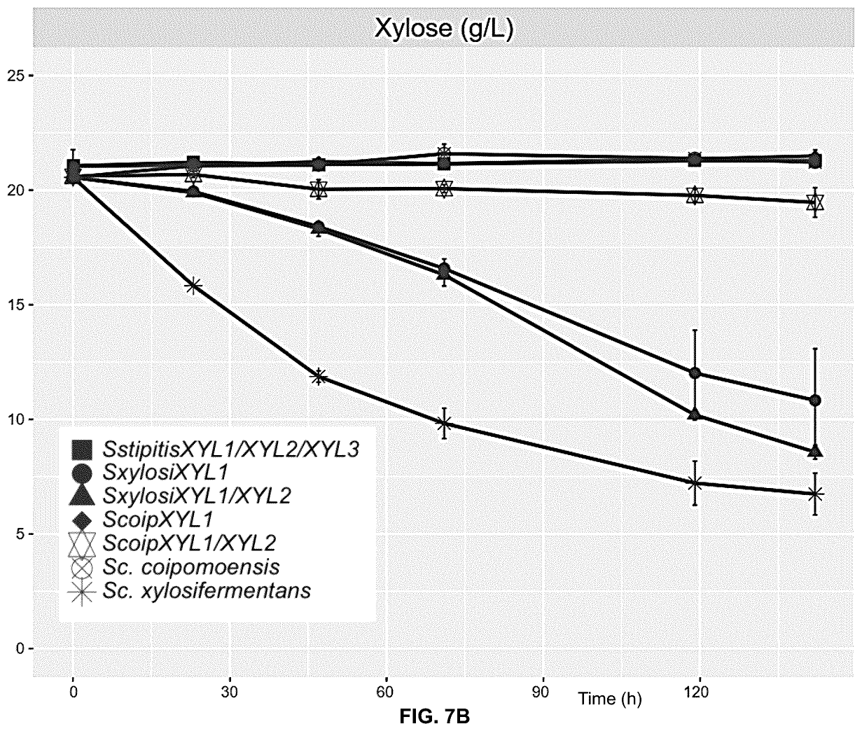

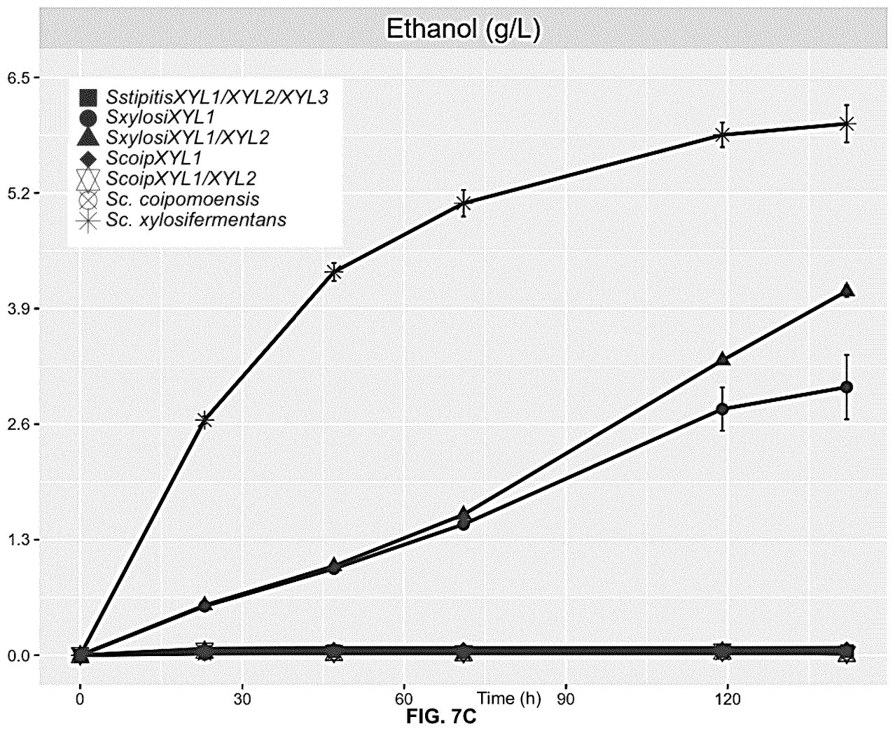

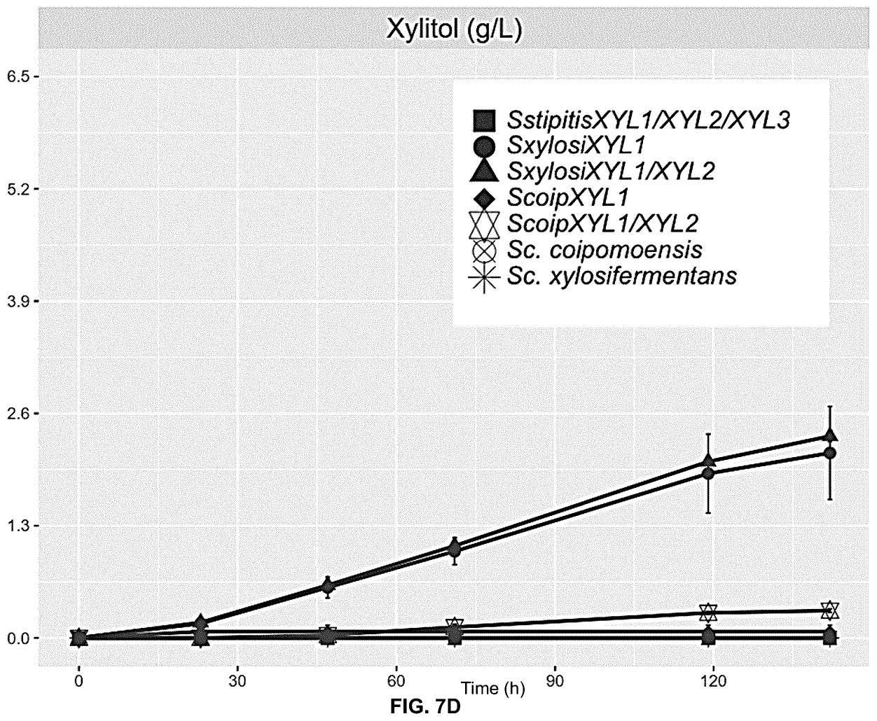

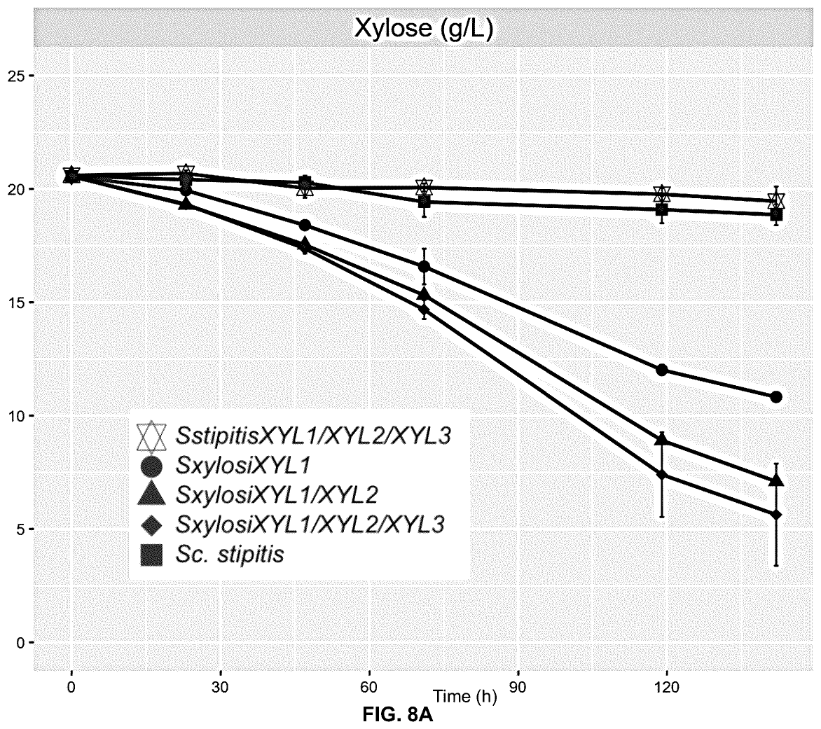

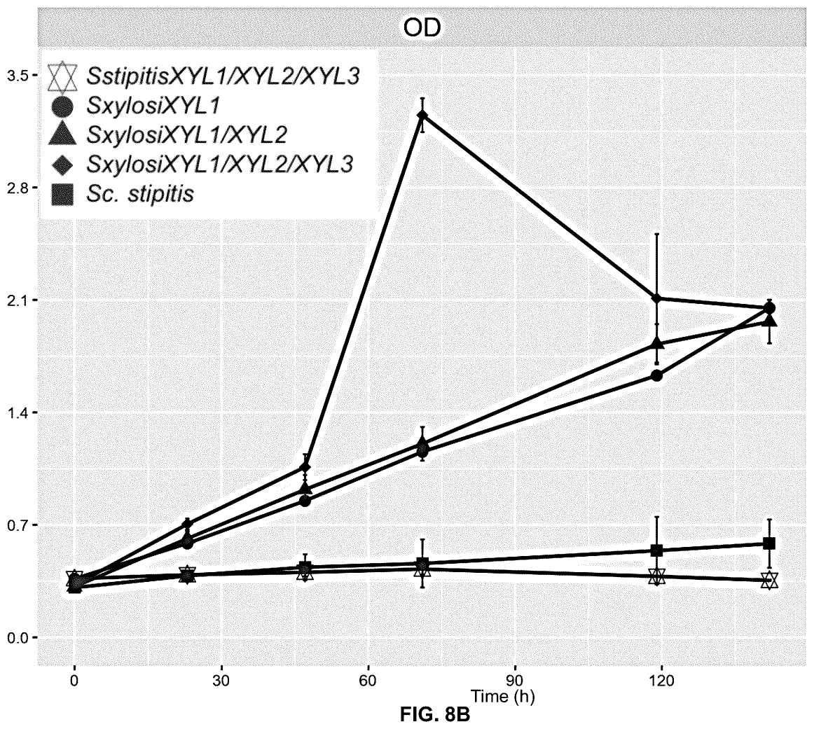

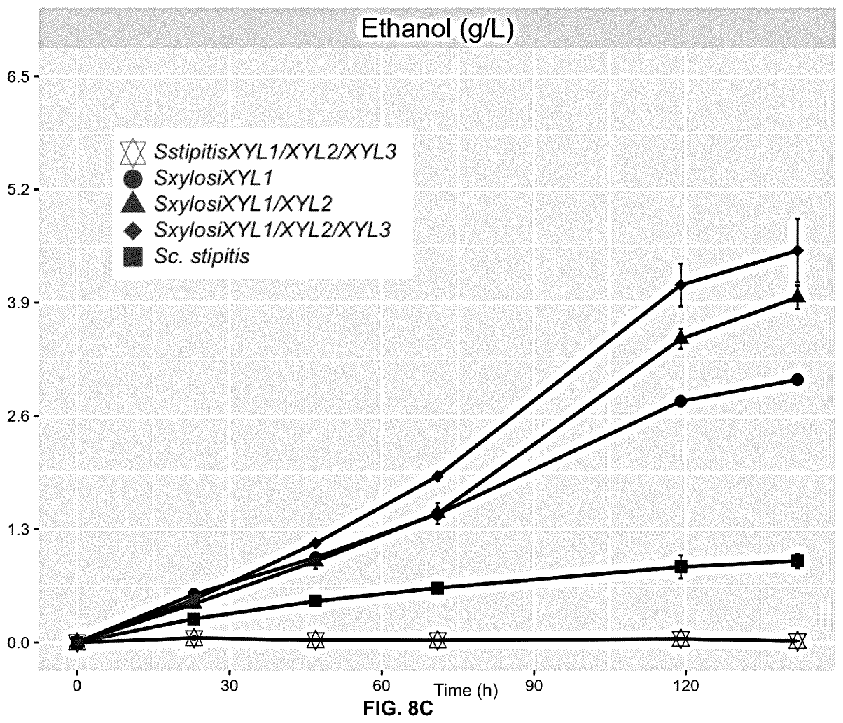

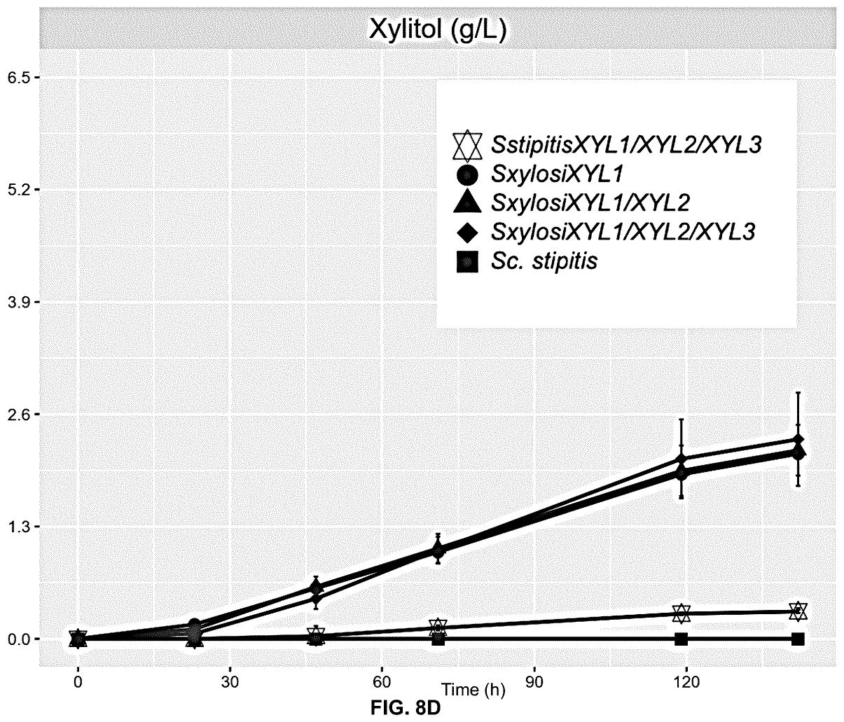

Our phenotypic results showed that Sc. xylosifermentans is a proficient xylose consumer, as it produced high levels of ethanol and rapidly consumed xylose. Sc. coipomoensis, on the other hand, accumulated high titers of xylitol. To test the possibility that the XR and XDH enzymes from Sc. xylosifermentans would enable greater xylose fermentation than Sc. coipomoensis enzymes, we used CRISPR-Cas9 to re-engineer a strain of S. cerevisiae that expresses Sc. stipites XYL1, XYL2, and XYL3. SstipitisXYL1 and SstipitisXYL2 were replaced with the corresponding genes from Sc. xylosifermentans (SxylosiXYL1 and SxylosiXYL2) and Sc. coipomoensis (ScoipXYL1 and ScoipXYL2). We hypothesized that the modified S. cerevisiae would grow faster anaerobically with Sc. xylosifermentans XYL1 because this XR enzyme lacked a cofactor preference (FIGS. 4A and 4B). In contrast, this would not be the case for the strain with XYL1 from Sc. coipomoensis or for the original parental strain containing SstipitisXYL1. Indeed, the strain expressing SxylosiXYL1 and SxylosiXYL1/XYL2 grew rapidly under anaerobic conditions compared to the strain SstipitisXYL1/XYL2/XYL3 (P<0.05), while the strains expressing ScoipXYL1 alone or ScoipXYL1/XYL2 presented an inferior growth profile (P<0.05) than the strain containing SstipitisXYL1/XYL2/XYL3 (FIG. 7A). Sc. stipites showed modest anaerobic growth with no production of ethanol and/or accumulation of intermediates (FIGS. 8A-8D). This result is interesting because the Sc. stipites XR can use both NADH and NADPH, but has a preference for the latter (36). Surprisingly, Sc. xylosifermentans grew anaerobically and produced ethanol, and did not excrete xylitol (FIGS. 7A-7D). Along with the other species and engineered strains, it also did not anaerobically produce glycerol, which is produced by S. cerevisiae under anoxic conditions to alleviate the cytosolic redox balance (37). Even though the replacement of Sc. stipites XYL genes with SxylosiXYL1/XYL2 improved xylose fermentation to ethanol by S. cerevisiae and enabled anaerobic growth, the mutants still excreted xylitol (FIG. 7D). This result contrasts what was observed natively with Sc. xylosifermentans, which did not excrete xylitol in anaerobic conditions.

Despite employing ATP instead of NAD(P)H as cofactors for the conversion of xylulose to xylulose-5-P, the xylulokinase enzyme encoded by XYL3 might contribute to xylitol accumulation due to the reversible nature of the previous reaction that generates xylulose from xylitol (38) (FIGS. 5A-5C). We hypothesized that XYL3 could be a reason for xylitol accumulation as well. We also engineered S. cerevisiae expressing Sc. xylosifermentans XYL3. Despite the ethanol production, xylose consumption, and growth by S. cerevisiae with SxylosiXYL1/XYL2/XYL3 were slightly improved compared to the strain carrying SxylosiXYL1 or SxylosiXYL1/XYL2 (P<0.05), suggesting that this gene is more active than Sc. stipites XYL3, but the strain still accumulated xylitol (FIGS. 8A-8D).

Finally, we confirmed that the XR and XDH enzymes maintained their predicted co-factor preferences when expressed in S. cerevisiae by performing enzymatic assays using whole cell lysates from the S. cerevisiae strains engineered with SxylosiXYL1/SxylosiXYL2 or ScoipXYL1/ScoipXYL2. Although the activities were lower compared to the native activities of XR in the donor species, XR encoded by SxylosiXYL1 in strain GLBRCY1847 (NADH: 1.97±0.01 U mg⁻¹; NADPH: 2.15±0.15 U mg⁻¹ P>0.05) still had fairly equal preferences between cofactors, while the XR encoded by ScoipXYL1 in GLBRCY1850 showed a preference for NADPH (NADH: 0.63±0.05 U mg⁻¹; NADPH: 1.18±0.03 U mg⁻¹, P<0.05). Xylitol dehydrogenase activity was high for NAD⁺ in both GLBRCY1847 (6.69±0.29 U mg⁻¹) and GLBRCY1850 (4.24±0.68 U mg⁻¹), and no activity was detected with NADP⁺.

### Discussion

To better understand the abilities of yeasts from the order Serinales to metabolize xylose, we quantified the metabolites produced by several species cultured in rich medium with xylose as the primary carbon source under high and moderate aeration conditions. Efficient xylose fermenters (ethanol producers) and inefficient xylose consumers (xylitol producers) were both identified. Most of the yeasts tested fermented xylose, but fewer than half primarily produced ethanol in SF. Instead, most yeasts mainly produced xylitol in the conditions tested. Xylitol, an intermediate of the xylose catabolism, is excreted by several species when a bottleneck in oxidation to xylulose occurs. Accumulation of xylitol in the extracellular medium was believed to occur due to the different XR and XDH cofactor requirements, which leads to cofactor imbalance under oxygen-limited (including moderate aeration) or anoxic conditions (15,39-41). Under these conditions, NADH accumulates, and NAD⁺ levels fall. Here, we unexpectedly observed that several species accumulated xylitol when sufficient oxygen was available, including species that were among the best ethanol producers in moderate oxygen-limiting conditions (FIGS. 1A-2B, Tables 1A-1C). This result strongly suggested that the different cofactor requirements by XR and XDH were not the sole issue limiting xylose fermentation.

An important question raised by our fermentation experiments was why the top xylose fermenting species, Sc. xylosifermentans, showed similar or better xylose fermentation than Sp. passalidarum in moderate aeration, even though the former has a single copy of XYL1, while the latter has two copies. Enzymatic assays were conducted to determine the activities and cofactor preferences of XR and XDH in each condition for key species. Assuming that redox imbalance primarily impacted xylose fermentation, our initial hypothesis was that the enzyme XR from species with high metabolic activity might present the same features as the XR encoded by XYL1.2 from Sp. passalidarum. In contrast, our findings revealed that the enzymes of all four species could use both NADH and NADPH as cofactors (FIGS. 4A and 4B), which was previously reported for Sc. stipites (36). The Sc. coipomoensis XR showed significant activity with NADH under moderate and high aeration, but it still had a preference for NADPH. An NADPH-dependent XR was also present in Sc. amazonensis, a species that also accumulated xylitol. Surprisingly, the XR of Sc. xylosifermentans did not have a cofactor preference. In this species, the lack of cofactor preference seems to naturally enhance flux for the second reaction of the xylose metabolism due to the oxidation of NADH to NAD⁺, which is the cofactor used in the proceeding step. Our results are consistent with a previous observation in which high aeration caused a switch the cofactor usage from NADH to NADPH for XR from Sp. passalidarum NRRL Y-27907 (23), and our transcriptome data showed that this switch likely occurs because the two paralogous XYL1 genes have different levels of expression in moderate and high aeration conditions (FIGS. 7A-7D). A similar result was reported by Cadete et al. (13), in which XYL1.2 was 15-16-fold higher expressed than XYL1.1 in severe oxygen-limiting conditions compared to moderate aeration. Collectively, these results suggest that XYL1.2 expression increases as the availability of oxygen decreases. Thus, Sp. passalidarum may use its second copy of XYL1, which encodes the NADH-preferring XR, to limit redox imbalance in low oxygen levels. The fast consumption of xylose and high yields of ethanol were not observed for other species that harbor multiple copies of XYL1, especially C. intermedia which was reported to have a NADH-dependent XR (19). Sp. passalidarum is unique among the species tested.

To elucidate the effect of oxygen on the expression profile of genes involved in the first steps of xylose metabolism and PPP, we determined the relative gene expression in Sc. amazonensis, Sc. coipomoensis, Sc. xylosifermentans and Sp. passalidarum under moderate and high aeration. The genes directly related to xylose metabolism (XYL1, XYL2, and XYL3) were all upregulated with moderate aeration (FIGS. 5A-5C) for Sc. xylosifermentans, which was supported by the higher XR and XDH activities observed in in vitro enzyme assays (FIGS. 4A-4C). Indeed, Sc. xylosifermentans XYL2 was among of the 15 most upregulated genes under oxygen limitation, the condition in which this species presented high levels of xylose consumption. Xylitol to xylulose conversion by XDH enzymes has been pointed as a limiting step for xylose fermentation, and high expression and codon optimization of XYL2 may be necessary for efficient xylose conversion (16,42,43). Even small expression changes may be associated with large phenotypic effects (44) and, in the case of Sc. xylosifermentans, XYL2 expression decreased significantly under high aeration. The xylitol accumulation by this species in this condition is likely in part due to this reduction in XYL2 expression, rather than the cofactor imbalances postulated for some other species.

Based on what is known about S. cerevisiae metabolism, the production of glycerol by Sp. passalidarum in high aeration is difficult to explain, especially considering that large amount it produced in BF. Glycerol can maintain redox balance in the absence of oxygen when ethanol flux is overloaded in S. cerevisiae (37). Strategies to increase glycerol production by yeasts include cutting off or attenuating ethanol production, shifting the NAD⁺/NADH ratio to increase the amount of NADH available, and overexpression of the glycerol 3-phosphate dehydrogenase GPD1 (45-47). The downregulation of PDC1 and ADH1, which encode pyruvate decarboxylase and alcohol dehydrogenase, respectively, in Sp. passalidarum and the upregulation of GPD1 and GPP1 (glycerol-3-phosphate phosphatase) under high aeration suggest a mechanism for the elevated glycerol production by this species in high aeration.

Genes from PPP, such as TKL1, TAL1, and RKI1, were also upregulated under moderate oxygen-limited condition for Sc. xylosifermentans (TKL1 and TAL1) and Sp. passalidarum (TKL1 and RKI1). Overexpression of those genes in engineered strains of S. cerevisiae improved the growth rate on xylose and the ethanol yield (48,49). ADH1 and ADH2 are responsible for ethanol production in Sc. stipites, and when the availability of oxygen becomes limited, the expression of ADH genes, especially ADH2, increases (50,51). While ADH1 was upregulated in the ethanol-producing yeasts under moderate aeration, ADH2 was the most upregulated gene for Sc. xylosifermentans, while it was differentially expressed in Sc. coipomoensis and Sc. amazonensis, but not Sp. passalidarum (FIGS. 5A-5C). In addition to the genes required for xylose metabolism, the high expression of the PPP and ethanol pathway in Sp. passalidarum and Sc. xylosifermentans might be related to the greater xylose metabolism by these species in moderate aeration.

Next, we genetically modified S. cerevisiae by the chromosomal integration of XYL genes from Sc. xylosifermentans and Sc. coipomoensis. Given the inability of S. cerevisiae to metabolize xylose, any observed growth must be attributed to the heterologously and constitutively expressed enzymes. Hence, this approach enabled us to investigate how redox balance influences anaerobic fermentation of xylose into ethanol in this model system. S. cerevisiae could grow in anaerobic conditions with SxylosiXYL1 and SxylosiXYL1/XYL2, but not with ScoipXYL1, ScoipXYL1/XYL2, and SstipitisXYL1/XYL2, suggesting that cofactor preference has important effects in anoxic conditions, at least when the pathway is constitutively expressed (FIGS. 7A-7D). S. cerevisiae strains containing SxylosiXYL1 and SxylosiXYL1/XYL2 produced ethanol, but they also accumulated xylitol. Swapping SstipitisXYL3 with SxylosiXYL3 only slightly relieved xylitol accumulation, so we hypothesize that other downstream and interacting native metabolic pathways in Sc. xylosifermentans may facilitate its xylitol-to-ethanol flux. Nonetheless, this experiment was essential to test the redox balance hypothesis, and it unexpectedly revealed that Sc. xylosifermentans was able to grow in anaerobic conditions, which are used in numerous industrial applications.

### Conclusions

We tested 30 species belonging to the order Serinales for xylose fermentation with a focus on the genera Spathaspora and Scheffersomyces and their close relatives. Collectively, our data reveal that xylose metabolism in the CUG-Ser1 clade is highly plastic and oxygen-dependent. Under high aeration conditions, several species switched from ethanol production to xylitol accumulation. This switch was generally accompanied by a decrease in enzyme activity and expression of genes related to xylose catabolism. While we find a global effect of oxygen availability on xylose metabolism, our data support the hypothesis that xylitol accumulation results from redox imbalance generated by differential cofactor preferences for XR and XDH in some species, but they also point to a novel role for oxygen-responsive gene regulation in other species that accumulate xylitol under high aeration, especially Sc. xylosifermentans. Among the species tested, Sc. xylosifermentans was also remarkable due to its high xylose consumption and ethanol formation under moderate aeration and even in anaerobic conditions, a phenomenon not previously noted for any xylose-fermenting yeast. This species may be a novel source of potential genes that can be expressed in industrial microbes like S. cerevisiae for biofuel production from lignocellulosic feedstocks. Alternatively, Sc. xylosifermentans could subjected to adaptive laboratory evolution or genetic modification to enhance its native potential and transform it into an industrial organism.

### Methods

### Yeast Strains and Growth Experiment Conditions

C. blattae, C. intermedia, Me. caribbica, Me. carpophila, Me. guilliermondii, and twenty-four strains of Scheffersomyces and Spathaspora species (Table 2) were obtained from the USDA Agricultural Research Service (ARS) NRRL Culture Collection in Peoria, Illinois, USA; Collection of Microorganisms and Cells of Universidade Federal de Minas Gerais, Belo Horizonte, Minas Gerais, Brazil; and CBS Yeast Collection of the Westerdijk Fungal Biodiversity Institute, Utrecht, the Netherlands.

TABLE 2
Yeast species used in this present examples and their respective isolation sources.

| Species | Strain | Source |
|---|---|---|
| Candida blattae | NRRL Y-27698^{T} | insect (Nguyen et al., 2007) |
| Candida intermedia | CBS 572^{T} | dairy (Kurtzman and Suzuki, 2010) |
| Meyerozyma caribbica | CBS 9966^{T} | insect (Vaughan-Martini et al., 2005) |
| Meyerozyma carpophila | CBS 5256^{T} | insect (Vaughan-Martini et al., 2005) |
| Meyerozyma guilliermondii | CBS 2030^{T} | insect frass (Kurtzman and Suzuki, |
| | | 2010) |
| Scheffersomyces amazonensis | UFMG-HMD-26.3^{T} | rotting wood (Cadete et al., 2012) |
| Scheffersomyces coipomoensis | NRRL Y-17651^{T} | rotten log (Urbina and Blackwell, |
| | | 2013) |
| Scheffersomyces cryptocercus | NRRL Y-48824^{T} | gut of wood cockroach (Urbina and |
| | | Blackwell, 2013) |
| Scheffersomyces ergatensis | NRRL Y-17652^{T} | larva of bark beetles (Urbina and |
| | | Blackwell, 2013) |
| Scheffersomyces gosingicus | CBS 11433^{T} | soil (Chang et al., 2011) |
| Scheffersomyces illinoinensis | NRRL Y-48827^{T} | rotten wood (Urbina and Blackwell, |
| | | 2012) |
| Scheffersomyces insectosa | NRRL Y-12854^{T} | Leptura maculicornis (Kurtzman and |
| | | Suzuki, 2010) |
| Scheffersomyces parashehatae | CBS 12535^{T} | larva of O. disjunctus (Suh et al., 2013) |
| Scheffersomyces quercinus | NRRL Y-48825^{T} | rotten wood (Urbina and Blackwell, |
| | | 2012) |
| Scheffersomyces shehatae | NRRL Y-12858^{T} | soil (Urbina and Blackwell, 2012) |
| Scheffersomyces spartinae | NRRL Y-7322^{T} | oyster grass (Kurtzman and Suzuki, |
| | | 2010) |
| Scheffersomyces stipites | NRRL Y-7124^{T} | beetle (Kurtzman and Suzuki 2010) |
| Scheffersomyces virginianus | NRRL Y-48822^{T} | rotten wood (Urbina and Blackwell, |
| | | 2012) |
| Scheffersomyces | CBS 12540^{T} | insect tunnel (Suh et al., 2013) |
| xylosifermentans | | |
| Spathaspora arborariae | UFMG-HMD-32.1 | rotting wood (Cadete et al., 2009) |
| Spathaspora brasiliensis | UFMG-HMD-19.3 | rotting wood (Cadete et al., 2013) |
| Spathaspora girioi | UFMG-CM-Y302^{T} | rotting wood (Lopes et al., 2016) |
| Spathaspora gorwiae | UFMG-CM-Y312^{T} | rotting wood (Lopes et al., 2016) |
| Spathaspora hagerdaliae | UFMG-CM-Y303^{T} | rotting wood (Lopes et al., 2016) |
| Spathaspora materiae | UFMG-07C151B^{T} | rotting wood (Barbosa et al., 2009) |
| Spathaspora passalidarum | NRRL Y-27907^{T} | rotting wood (Nguyen et al., 2011) |
| Spathaspora roraimanensis | UFMG-HMD-23.2^{T} | rotting wood (Cadete et al., 2013) |
| Spathaspora suhii | UFMG-CM-Y475^{T} | rotting wood (Cadete et al., 2013) |
| Spathaspora xylofermentans | UFMG-CM-Y478^{T} | rotting wood (Cadete et al., 2013) |

The GLBRCY38 strain was generated from a haploid spore from the diploid GLBRCY2A strain (52) containing an integrated DNA cassette to express Sc. stiptis XYL1, XYL2 and XYL3 genes in the HO locus (18). GLBRCY2A was sporulated and individual tetrads were dissected as previously described (53). Haploid spores were verified for the XYL cassette by PCR and selection for the kanMX resistance marker. One spore containing the XYL cassette, GLBRCY25A, was selected and all XYL gene sequences were fully confirmed for accuracy by PCR and Sanger sequencing. The kanMX marker was excised (54) to generate the GLBRCY38 strain. We used CRISPR-Cas9 to swap the genes XYL1 and XYL2 to the correspondent genes from Sc. xylosifermentans (SxylosiXYL1 and SxylosiXYL2) and Sc. coipomoensis (ScoipXYL1 and ScoipXYL2) in the strain GLBRCY38 with gRNA expression plasmid (pXIPHOS) (55) targeting XYL1 (CAACAGCCAAAACCCACGGC (SEQ ID NO:10)) or XYL2 (CTTAACCAAGAAATCTTCGG (SEQ ID NO:11)). Transformation of yeast strains was done using the lithium acetate/PEG-4000/carrier DNA method (56). After the transformation, the colonies were selected by plating them on YPD agar with 100 mg/L nourseothricin (NAT). All strains were confirmed for gene swaps and antibiotic marker excision by PCR with gene-specific primers or flanking primers. Sanger sequencing of purified PCR products was performed by the University of Wisconsin-Madison Biotechnology Center. The engineered strains used in this work are summarized in Table 3.

TABLE 3
S. cerevisiae strains and their genotypes
used in the present examples.

| Genotype | Strain names |
|---|---|
| NRRL YB-210^{MR} MATa spore with | GLBRCY38 |
| HOΔ::SstipitisXYL1\|XYL2\|XYL3 | |
| GLBRCY38 with SstipitisXYL1Δ::SxylosiXYL1 | GLBRCY1840 |
| GLBRCY1840 with SstipitisXYL2Δ::SxylosiXYL2 | GLBRCY1847 |
| GLBRCY1847 with SstipitisXYL3Δ::SxylosiXYL3 | GLBRCY1866 |
| GLBRCY38 with SstipitisXYL1Δ::ScoipXYL1 | GLBRCY1843 |
| GLBRCY1843 with SstipitisXYL2Δ::ScoipXYL2 | GLBRCY1850 |

Strains from all yeast species were initially plated from freezer stock on 10 g/L yeast extract, 20 g/L peptone and 2% dextrose (YPD) plates and grown for single colonies. Single colonies of each strain were pre-cultured in 10 mL of YPX medium (1% yeast extract, 2% peptone, 2% xylose) overnight at 30° C. under 200 rpm. Cells were recovered by centrifugation at 2600 g for 10 min, washed with sterile water, and suspended in the fermentation medium at 0.5 g dry cell weight L⁻¹ of cell concentration. To evaluate the performance of the yeasts in different aeration conditions, we used 125-mL shake-flasks (SF) for moderate aeration, and 250-mL baffled-flasks (BF) for high aeration, both containing 50 mL of YPX (1% yeast extract, 2% peptone, 5% xylose). Fermentation assays were also carried out in 125-mL SF with 125 mL of YPX (low aeration) to test the behavior of some yeasts in low oxygenation availability. The dissolved oxygen available in each flask was measured by a dissolved oxygen meter (Mettler Toledo F4-Field FiveGo, USA). The flasks were incubated at 30° C. under 200 rpm for 72 h. Cell growth was determined by collecting 1 mL of the culture. The cells were recovered by centrifugation and dried in a speed vacuum concentrator. The ethanol, xylitol, and biomass yields (Yp/s^{et}, Yp/s^{xy}, Yx/s, g g⁻¹), volumetric productivity of ethanol and xylitol (Qp^{et}, Qp^{xy}, g·L⁻¹·h⁻¹), and consumption of D-xylose were determined as described previously by Cadete et al. (13).

Growth experiment of engineered strains was carried out with 125 mL-baffled flasks in anaerobic chamber for 142 h. Sc. xylosifermentans, Sc. coipomoensis, and Sc. stipites were also tested under this condition. The pre-culture was done exactly as described above, but for inoculation, we shifted into flasks containing 30 mL YPX media (2% xylose) at a concentration of optical density at λ=600 nm (OD₆₀₀)=0.3. Seven-hundred microliters of sample were taken during the experiment for measuring the OD₆₀₀ and for quantifying end-products using high-performance liquid chromatography (HPLC) and refractive index detection (RID). We performed Student's t-tests to determine if there were significant differences (P<0.05) between the parental strain and engineered strains.

### HPLC-RID Analysis

Seven-hundred microliters of samples for end-product analysis were collected throughout the fermentation, centrifuged at 13500×g for 5 minutes, and supernatants were stored at −20° C. for analysis. Glucose, xylose, glycerol, xylitol, succinic acid, lactic acid, formic acid, acetic acid, pyruvate, cellobiose, and ethanol were separated by HPLC and subsequently quantified using refractive index detection. HPLC-RID was conducted with an Agilent 1260 Infinity system (Agilent Technologies, Palo Alto, CA) equipped with an Aminex HPX-87H anion-exchange column, 300 by 7.8 mm (Bio-Rad, Hercules, CA). Samples were diluted with 9 volumes of H₂O, injected into the HPLC-RID system (50-1 injection volume), and eluted isocratically with 0.02 N H₂SO₄ at a flow rate of 0.5 mL·min⁻¹ (RID flow cell, 45° C.; column, 50° C.). Reference compounds (Thermo Fisher) were diluted in H₂O and used to generate a standard curve. Analyte concentrations were calculated using Chem Station software version B.04.03 (Agilent Technologies) (57). Plots were constructed in R v3.6.3 using the Rstudio v1.3.1073 platform.

### Enzyme Activities

Yeast species Sc. xylosifermentans, Sc. coipomoensis, and Sc. amazonensis were grown in YPX medium as described above or in YPD for the negative control, using both SF and BF. Engineered strains Y1847 and Y1850 were also tested, but they grew in 125-mL SF with 30 mL YPX (2% xylose) in aerobic conditions. Cells were harvested at mid-log growth phase, washed with cold sterile water, and extracted with Y-PER® Yeast Protein Extraction Reagent (Thermo Fisher). Protein concentrations from the crude cell extracts were determined by BCA Protein Assay Kit (Thermo Fisher). XR activities were obtained from 250 μL reactions containing 100 mM triethanolamine buffer pH 8, 0.2 mM NADPH or NADH, 0.2 M D-xylose, crude cell extract, and deionized water; XDH activities were obtained from 250 μL reactions containing 100 mM glycine buffer pH 9, 50 mM MgCl₂, 3 mM NADP⁺ or NADP⁺, 0.2 M xylitol, crude cell extract, and deionized water (13). Enzyme activities were determined by oxidation or reduction of NADH/NADPH or NAD⁺/NADP⁺, respectively. Reaction mixtures aliquoted into 96-well microtiter plates (Corning® 96 Well Clear Flat Bottom UV-Transparent, Darmstadt, Germany) were placed in Tecan® (Infinite M-1000, Switzerland) at 25° C. for measuring absorbance at 340 nm for 1 h. Standard curves of NADPH and NADH were used to calculate the concentration of the samples. extracted proteins from the yeast Sp. passalidarum were used as positive control, while S. cerevisiae 288SC was used as a negative control. In addition, blank measurements with samples lacking either cell lysate or xylose substrate were performed for each sample, and the resulting values were subtracted from the test values. Enzyme activities were determined from three independent biological replicates. The specific activity of each enzyme was estimated by the number of enzyme units per mL divided by the concentration of protein in mg/mL. One unit was defined as the generation of 1 μmol NAD(P)H or NAD(P)⁺ per min. We performed paired Student's t-tests to determine if there were significant differences (P<0.05) between NADH and NADPH usage by XR from each species. Data analyses and plots were performed in R v3.6.3 using the Rstudio v1.3.1073 platform.

### Genome Extraction, Sequencing, Assembly, and Annotation

Genomic DNA (gDNA) of the species Sc. xylosifermentans, Sc. amazonensis, and Sc. coipomoensis was isolated using a modified phenol:chloroform method (58). The sequencing was performed at the DOE Joint Genome Institute Standard. Genome sequencing was performed using Pacific Biosciences (PacBio) Multiplexed >10 kb with Blue Pippin Size Selection (AMPure Beads for Sc. coipomoensis). Filtered subread data was processed with the JGI quality control pipeline to remove artifacts. The mitochondrial genome was assembled separately with the circular consensus sequencing (CCS) reads and polished with two rounds of RACON version 1.4.13 (59). The mitochondria-filtered CCS reads were then assembled with Flye version 2.8.1 (60) to generate an assembly and polished with two rounds of RACON version 1.4.13. The Sc. coipomoensis nuclear genome was assembled using Falcon v. 1.8.8 (61), improved with FinisherSC, and polished with Arrow version SMRTLink v5.0.1.9578 (62). Ribosomal DNA (rDNA) was assembled separately from a subset of CCS reads identified using kmer matching against the UNITE database with BBTools (sourceforge.net/projects/bbmap) version 38.79. Matching reads were subsampled to 600000 bp with BBTools and assembled with Flye version 2.8.1 and polished with two rounds of RACON version 1.4.13. The eukaryotic internal transcribed spacer (ITS) was identified and extracted from the rDNA assembly using ITSx (Bengtsson-Palme et al., 2013). Results were used to orient and trim the rDNA contig to 100 bp SSU—1 Kb LSU. Contigs less than 1000 bp were excluded. Completeness of the euchromatic portion of the genome assembly was assessed by aligning assembled consensus RNA sequence data with BBTools. Genomes were then annotated using the JGI annotation pipeline (63). Assembly and annotation statistics for the genomes sequenced are summarized in Table 4.

TABLE 4
Assembly and annotations statistics for the
genomes sequenced for the present examples.

| Lineage | Sc. xylosifermentans | Sc. coipomoensis | Sc. amazonensis |
|---|---|---|---|
| # Contigs | 11 | 23 | 9 |
| Assembly length (Mbp) | 17.25 | 14.76 | 14.21 |
| N50 (Mbp) | 2.38 | 2.31 | 2.01 |
| % repeats | 1.87 | 4.02 | 5.04 |
| # gene models | 6180 | 6180 | 6071 |
| % CDS complete | 98.59 | 98.58 | 98.35 |
| % with PFAM | 81.36 | 79.53 | 80.7 |
| % BUSCO* | 99.6 | 98.6 | 99.1 |

Mbp = Megabasepairs.
*Percent of BUSCO (v5.0) genes from the saccharomyces_odb10 database present (including single-copy, duplicated and fragmented genes) in each genome.

### RNA Extraction, Sequencing, and Data Analysis

Cells from Sp. passalidarum, Sc. xylosifermentans, Sc. coipomoensis, and Sc. amazonensis were grown on YPD agar for 48 h to single colonies, which were inoculated into 10 mL of YPX medium overnight into 50 mL glass tube at 30° C. After pre-culture, aliquots were transferred to 125 mL SF and 250 mL BF, both flasks with 50 mL of YPX, at an initial of inoculum of 0.5 g L⁻¹, and incubated at 30° C. under 200 rpm until mid-log phase. RNA was extracted using the acid phenol protocol (64). Briefly, the total volume of the cultures was centrifuged with 5% phenol and 95% EtOH, and it was flash-frozen in a dry ice-ethanol bath. Cells were resuspended in TES buffer lysis (10 mM Tris, 10 mM EDTA, 0.5% SDS in water) plus PVP40, acid-washed beads (Sigma #G8772), and one volume of saturated acid phenol (IBI Scientific). Lysates were incubated at 65° C. for one hour with vortexing every 15 minutes. Then, lysates were extracted with 1 volume of acid phenol each and once with 1 volume of chloroform. The aqueous phase of the final chloroform extraction was removed and added to a solution consisting of 2.5 volumes of 95%-100% ethanol and 0.1 volumes of 3 M sodium acetate and the tubes were placed at −80° C. overnight to precipitate the RNA. RNA pellets were then collected by centrifugation, washed twice in 70% ethanol each, and resuspended in Rnase-free water. Purified RNA was then treated with Dnase I (NEB #EN0521) to remove any residual DNA prior to treatment with the RNA Clean & Concentrator kit (Qiagen #74134). Total RNA yields were quantified with the Qubit RNA Assay Kit (Thermo Fisher).

mRNA library preparation, quantification, and sequencing were performed at the DOE Joint Genome Institute. Paired-end libraries were sequenced on Illumina NovaSeq S4 (2×151). Raw FASTQ file reads were trimmed to remove adapters and artifact sequences using Trimmomatic version 0.30 (65). The filtered reads of each species were aligned to their respective reference genome with Bowtie2 version 2.4.5 (66) with average mapping rates of 99.7, 98.8, 98.8, and 98.7% for Sp. passalidarum, Sc. xylosifermentans, Sc. coipomoensis, and Sc. amazonensis, respectively. featureCounts (67) was used to generate the raw gene counts. Raw sequencing reads were normalized using the reads per kilobase per million mapped reads (RPKM). Deseq2 version 1.38.1 (68) was used to perform quality control analysis and identify significantly differentially expressed genes (DEGs) from pairwise analyses with the raw counts, using Benjamini-Hochberg false discovery rate (FDR) of less than 0.05 as a significance threshold and log 2 fold change >1 or <−1 for differentiating between up and down regulated genes, respectively. The enrichment analysis was done by mapping the gene ontology (GO) terms with the gene identifiers, then performing Fisher's exact test and Benjamini-Hochberg-corrected tests using a threshold of 0.05 with SciPy version 1.9.0 (69) and Python Statsmodels version 0.10.2 (70).

For genome annotation, Sc. xylosifermentans, Sc. amazonensis, and Sc. coipomoensis filtered RNASeq reads were also assembled de-novo with Trinity v.2.3.2 using -normalize_reads and -jaccard_clip options (71).

### Data Availability

Raw RNA-Seq and PacBio genome reads of Sc. xylosifermentans CBS 12540^{T}, Sc. coipomoensis NRRL Y-17651^{T}, and Sc. amazonensis UFMG-HMD-26.3^{T} are available on the JGI portal genome.jgi.doe.gov. The reference genome of Sp. passalidarum NRRL Y-27907^{T} is publicly available (18). The genome assemblies and annotations of Sc. xylosifermentans, Sc. amazonensis, and Sc. coipomoensis are available from JGI fungal genome portal MycoCosm (63); (mycocosm.jgi.doe.gov).

### REFERENCES

1. Liu Y, Cruz-Morales P, Zargar A, Belcher M S, Pang B, Englund E, et al. Biofuels for a sustainable future. Cell. 2021; 184(6):1636-47.
2. Zhao Z, Xian M, Liu M, Zhao G. Biochemical routes for uptake and conversion of xylose by microorganisms. Biotechnology for Biofuels. 2020, 13:1-2.
3. Parapouli M, Vasileiadis A, Afendra A S, Hatziloukas E. Saccharomyces cerevisiae and its industrial applications. AIMS Microbiology. 2020, 6:1-31.
4. Hittinger C T. Saccharomyces diversity and evolution: A budding model genus Trends in Genetics 2013, 29(5):309-17.
5. Lee S B, Tremaine M, Place M, Liu L, Pier A, Krause D J, et al. Crabtree/Warburg-like aerobic xylose fermentation by engineered Saccharomyces cerevisiae Metabolic engineering. 2021, 68:1096-7176.
6. Osiro K O, Borgström C, Brink D P, Fjölnisdöttir BL, Grauslund M F G. Exploring the xylose paradox in Saccharomyces cerevisiae through in vivo sugar signalomics of targeted deletants. Microbial Cell Factories. 2019, 18: 1-19.
7. Selim K A, Easa S M, El-Diwany A I. The Xylose Metabolizing Yeast Spathaspora passalidarum is a Promising Genetic Treasure for Improving Bioethanol Production. Fermentation. 2020; 6(1):33.
8. Groenewald M, Hittinger C T, Bensch K, Opulente D A, Shen X X, Li Y, et al. A genome-informed higher rank classification of the biotechnologically important fungal subphylum Saccharomycotina. Studies in Mycology. 2023; 105(1):1-22.
9. Shen X X, Opulente D A, Kominek J, Zhou X, Steenwyk J L, Buh K V., et al. Tempo and Mode of Genome Evolution in the Budding Yeast Subphylum. Cell. 2018; 175(6):1533-1545.e20.
10. Barros K O, Alvarenga F B M, Magni G, Souza G F L, Abegg M A, Palladino F, et al. The Brazilian Amazonian rainforest harbors a high diversity of yeasts associated with rotting wood, including many candidates for new yeast species. Yeast. 2022, 40(2):84-101.
11. Cadete R M, Rosa C A. The yeasts of the genus Spathaspora: potential candidates for second-generation biofuel production. Yeast. 2018; 35(2):191-9.
12. Urbina H, Blackwell M. Multilocus phylogenetic study of the Scheffersomyces yeast clade and characterization of the N-terminal region of xylose reductase gene. PloS One. 2012; 7(6): e39128.
13. Kurtzman C P, Suzuki M. Phylogenetic analysis of ascomycete yeasts that form coenzyme Q-9 and the proposal of the new genera Babjeviella, Meyerozyma, Millerozyma, Priceomyces, and Scheffersomyces. Mycoscience. 2010, 51(1):2-14.
14. Nguyen N H, Suh S O, Marshall C J, Blackwell M. Morphological and ecological similarities: wood-boring beetles associated with novel xylose-fermenting yeasts, Spathaspora passalidarum gen. sp. Nov. and Candida jeffriesii sp. Nov. Mycological research. 2006, 110(10):1232-41.
15. Cadete R M, Heras A M De, Sandström AG, Ferreira C, Gírio F, Françoise M, et al. Exploring xylose metabolism in Spathaspora species: XYL1.2 from Spathaspora passalidarum as the key for efficient anaerobic xylose fermentation in metabolic engineered Saccharomyces cerevisiae. Biotechnology for Biofuels. 2016, 9(1):1-4.
16. Jeffries T W, Jin Y S. Metabolic engineering for improved fermentation of pentoses by yeasts. Applied Microbiology and Biotechnology. 2004, 63:495-509.
17. Jeffries T W, Grigoriev I V, Grimwood J, Laplaza J M, Aerts A, Salamov A, et al. Genome sequence of the lignocellulose-bioconverting and xylose-fermenting yeast Pichia stipites. Nature Biotechnology. 2007, 25(3):319-26.
18. Wohlbach D J, Kuo A, Sato T K, Potts K M, Salamov A A, LaButti K M, et al. Comparative genomics of xylose-fermenting fungi for enhanced biofuel production. Proceedings of the National Academy of Sciences. 2011,108(32):13212-7.
19. Geijer C, Faria-Oliveira F, Moreno A D, Stenberg S, Mazurkewich S, Olsson L. Genomic and transcriptomic analysis of Candida intermedia reveals the genetic determinants for its xylose-converting capacity. Biotechnology for Biofuels. 2020, 13:1-5.
20. Jeppsson M, Bengtsson O, Franke K, Lee H, Hahn-Hägerdal B, Gorwa-Grauslund M F. The expression of a Pichia stipites xylose reductase mutant with higher KM for NADPH increases ethanol production from xylose in recombinant Saccharomyces cerevisiae. Biotechnology and Bioengineering. 2006, 93(4):665-73.
21. Leitgeb S, Petschacher B, Wilson D K, Nidetzky B. Fine tuning of coenzyme specificity in family 2 aldo-keto reductases revealed by crystal structures of the Lys-274→Arg mutant of Candida tenuis xylose reductase (AKR2B5) bound to NAD+ and NADP+. FEBS Lett. 2005, 579(3):763-7.
22. Kim S R, Kwee N R, Kim H, Jin Y S. Feasibility of xylose fermentation by engineered Saccharomyces cerevisiae overexpressing endogenous aldose reductase (GRE3), xylitol dehydrogenase (XYL2), and xylulokinase (XYL3) from Scheffersomyces stipites. FEMS Yeast Research, 13(3): 312-321.
23. Bonan CIDG, Biazi L E, Dionísio SR, Soares L B, Tramontina R, Sousa A S, et al. Redox potential as a key parameter for monitoring and optimization of xylose fermentation with yeast Spathaspora passalidarum under limited-oxygen conditions. Bioprocess Biosyst Eng [Internet]. 2020; 43(8):1509-19.
24. Hou X. Anaerobic xylose fermentation by Spathaspora passalidarum. Applied Microbiology and Biotechnology. 2012, 94:205-14.
25. Borelli G, Fiamenghi M B, Dos Santos L V, Carazzolle M F, Pereira G A G, José J, et al. Positive Selection Evidence in Xylose-Related Genes Suggests Methylglyoxal Reductase as a Target for the Improvement of Yeasts' Fermentation in Industry. Genome Biology and Evolution. 2019, 11(7):1923-38.
26. Bengtsson O, Hahn-Hägerdal B, Gorwa-Grauslund M F. Biotechnology for Biofuels Xylose reductase from Pichia stipites with altered coenzyme preference improves ethanolic xylose fermentation by recombinant Saccharomyces cerevisiae. Biotechnology for Biofuels. 2009; 2:1-0.
27. Kim S R, Park Y C, Jin Y S, Seo J H. Strain engineering of Saccharomyces cerevisiae for enhanced xylose metabolism. Biotechnology Advances. 2013, 31(6):851-61.
28. Nogueira Moysés D, Castelo Branco Reis V, Ricardo Moreira de Almeida J, Maria Pepe de Moraes L, Araripe Gonçalves Torres F. Xylose Fermentation by Saccharomyces cerevisiae: Challenges and Prospects. International Journal of Molecular Sciences. 2016; 17(3): 207.
29. Cunha J T, Soares P O, Romaní A, Thevelein J M, Domingues L. Xylose fermentation efficiency of industrial Saccharomyces cerevisiae yeast with separate or combined xylose reductase/xylitol dehydrogenase and xylose isomerase pathways. Biotechnology for Biofuels. 2019; 12(1): 1-14.
30. Brink D P, Borgström C, Persson V C, Osiro K O, Gorwa-Grauslund M F. D-xylose sensing in Saccharomyces cerevisiae: Insights from D-glucose signaling and native D-xylose utilizers. International Journal of Molecular Sciences. 2021, 22(22):12410.
31. Ndubuisi I A, Amadi C O, Nwagu T N, Murata Y, Ogbonna J C. Non-conventional yeast strains: Unexploited resources for effective commercialization of second-generation bioethanol. Biotechnology Advances. 2023, 17:108100.
32. Guo J, Huang S, Chen Y, Guo X, Xiao D. Heterologous expression of Spathaspora passalidarum xylose reductase and xylitol dehydrogenase genes improved xylose fermentation ability of Aureobasidium pullulans. Microbial Cell Factories. 2018, 17(1):1-1.
33. Zhu Y, Zhang J, Zhu L, Jia Z, Li Q, Xiao W, et al. Minimize the Xylitol Production in Saccharomyces cerevisiae by Balancing the Xylose Redox Metabolic Pathway. Frontiers in Bioengineering and Biotechnology. 2021, 26(9):639595.
34. Xie C Y, Yang B X, Song Q R, Xia Z Y, Gou M, Tang Y Q. Different transcriptional responses of haploid and diploid S. cerevisiae strains to changes in cofactor preference of XR. Microbial Cell Factories. 2020, 19(1):1-6.
35. Papini M, Nookaew I, Uhlén M, Nielsen J. A comparative systems biology study with the Crabtree positive yeast Saccharomyces cerevisiae. Microbial Cell Factories. 2012, 11:1-16.
36. Bruinenberg P M, De But P H M, Van Dijken J P, Scheffers W A. NADH-linked aldose reductase: the key to anaerobic alcoholic fermentation of xylose by yeasts. Applied Microbiology and Biotechnology. 1984, 19:256-60.
37. Jain V K, Divol B, Prior B A, Bauer F F. Elimination of glycerol and replacement with alternative products in ethanol fermentation by Saccharomyces cerevisiae. Journal of Industrial Microbiology and Biotechnology. 2011, 38(9):1427-35.
38. Veiga L. Polyol dehydrogenases in Candida albicans II. Xylitol oxidation to D-xylulose. The Journal of General and Applied Microbiology. 1968; 14(1):79-87.
39. Trichez D, Steindorff A S, Soares CEVF, Formighieri E F, Almeida J R M. Physiological and comparative genomic analysis of new isolated yeasts Spathaspora sp. JA1 and Meyerozyma caribbica JA9 reveal insights into xylitol production. FEMS Yeast Research. 2019, 19(4):1-15.
40. Veras H C T, Parachin N S, Almeida J R M. Comparative assessment of fermentative capacity of different xylose-consuming yeasts. Microbial Cell Factories. 2017; 16(1):1-8.
41. Cadete R M, Melo-Cheab M A, Viana A L, Oliveira E S, Fonseca C, Rosa C A. The yeast Scheffersomyces amazonensis is an efficient xylitol producer. World Journal of Microbiology and Biotechnology. 2016, 32:1-5.
42. Nalabothu R L, Fisher K J, LaBella A L, Meyer T A, Opulente D A, Wolters J F, et al. Codon optimization, not gene content, predicts XYLose metabolism in budding yeasts. bioRxiv. 2022 June 13:2022-06.
43. Kim S R, Ha S J, Kong I I, Jin Y S. High expression of XYL2 coding for xylitol dehydrogenase is necessary for efficient xylose fermentation by engineered Saccharomyces cerevisiae. Metabolic Engineering. 2012, 14(4):336-43.
44. Lopes-Ramos C M, Chen C Y, Kuijjer M L, Paulson J N, Sonawane A R, Fagny M, et al. Sex Differences in Gene Expression and Regulatory Networks across 29 Human Tissues. Cell Reports. 2020, 31(12).
45. Semkiv M V., Ruchala J, Dmytruk K V., Sibirny A A. 100 Years Later, What Is New in Glycerol Bioproduction? Trends in Biotechnology. 2020, 38(8):907-16.
46. Murashchenko L, Abbas C, Dmytruk K, Sibirny A, Sibirny A. Overexpression of the truncated version of ILV2 enhances glycerol production in Saccharomyces cerevisiae. Yeast. 2016, 33(8):463-9.
47. Michnick S, Roustan J L, Remize F, Barre P, Dequin S. Modulation of Glycerol and Ethanol Yields During Alcoholic Fermentation in Saccharomyces cerevisiae Strains Overexpressed or Disrupted for GPD1 Encoding Glycerol 3-Phosphate Dehydrogenase. Yeast. 1997, 13(9):783-93.
48. Kobayashi Y, Sahara T, Ohgiya S, Kamagata Y, Fujimori K E. Systematic optimization of gene expression of pentose phosphate pathway enhances ethanol production from a glucose/xylose mixed medium in a recombinant Saccharomyces cerevisiae. AMB Express. 2018, 8:1-1.
49. Diao L, Liu Y, Qian F, Yang J, Jiang Y, Yang S. Construction of fast xylose-fermenting yeast based on industrial ethanol-producing diploid Saccharomyces cerevisiae by rational design and adaptive evolution. BMC Biotechnology. 2013, 13(1):1-9.
50. Papini M, Nookaew I, Uhlén M, Nielsen J. Scheffersomyces stipites: A comparative systems biology study with the Crabtree positive yeast Saccharomyces cerevisiae. Microbial Cell Factories. 2012, 11:1-6.
51. Passoth V, Schäfer B, Liebel B, Weierstall T, Klinner U. Molecular cloning of alcohol dehydrogenase genes of the yeast Pichia stipites and identification of the fermentative ADH. Yeast. 1998, 14(14):1311-25.
52. Sato T K, Liu T, Parreiras L S, Williams D L, Wohlbach D J, Bice B D, et al. Harnessing genetic diversity in Saccharomyces cerevisiae for fermentation of xylose in hydrolysates of alkaline hydrogen peroxide-pretreated biomass. Applied and Environmental Microbiology. 2014 Jan. 15; 80(2):540-54.
53. Parreiras L S, Breuer R J, Narasimhan R A, Higbee A J, La Reau A, Tremaine M, et al. Engineering and two-stage evolution of a lignocellulosic hydrolysate-tolerant Saccharomyces cerevisiae strain for anaerobic fermentation of xylose from afex pretreated corn stover. PloS One. 2014, 9(9):e107499.
54. Güldener U, Heck S, Fiedler T, Beinhauer J, Hegemann J H. A new efficient gene disruption cassette for repeated use in budding yeast. Nucleic Acids Research. 1996, 24(13):2519-24.
55. Higgins D A, Young M K M, Tremaine M, Sardi M, Fletcher J M, Agnew M, et al. Natural Variation in the Multidrug Efflux Pump SGE1 Underlies Ionic Liquid Tolerance in Yeast. Genetics. 2018, 210(1):219-34.
56. Woods R, Gietz R. High-efficiency transformation of plasmid DNA into yeast. In: MacDonald PN, editor. Two-hybrid systems. Totowa: Humana Press Inc.; 2001. P. 85-98.
57. Schwalbach M S, Keating D H, Tremaine M, Marner W D, Zhang Y, Bothfeld W, et al. Complex physiology and compound stress responses during fermentation of alkali-pretreated corn stover hydrolysate by an Escherichia coli ethanologen. Applied and Environmental Microbiology. 2012, 78(9):3442-57.
58. Moolhuijzen P, See P T, Moffat C S. A new PacBio genome sequence of an Australian Pyrenophora tritici-repentis race 1 isolate. BMC Research Notes. 2019, 12:1-5.
59. Vaser R, Sović I, Nagarajan N, Šikić M. Fast and accurate de novo genome assembly from long uncorrected reads. Genome Research. 2017, 27(5):737-46.
60. Kolmogorov M, Yuan J, Lin Y, Pevzner P A. Assembly of long, error-prone reads using repeat graphs. Nature Biotechnology. 2019, 37(5):540-6.
61. Chin C S, Alexander D H, Marks P, Klammer A A, Drake J, Heiner C, et al. Nonhybrid, finished microbial genome assemblies from long-read smrt sequencing data. Nature methods. 2013, 10(6):563-9.
62. Lam K K, Labutti K, Khalak A, Tse D. FinisherSC: A repeat-aware tool for upgrading de novo assembly using long reads. Bioinformatics. 2015, 31(19):3207-9.
63. Grigoriev I V., Nikitin R, Haridas S, Kuo A, Ohm R, Otillar R, et al. MycoCosm portal: Gearing up for 1000 fungal genomes. Nucleic Acids Research. 2014, 42(D1):D699-704.
64. Chomczynski P, Sacchi˜ N. Isolation by Acid Guanidinium Thiocyanate-Phenol-Chloroform Extraction. Analytical Biochemistry. 1987, 162(1):156-9.
65. Bushnell B. BBMap: a fast, accurate, splice-aware aligner. Berkeley: Lawrence Berkeley National Lab. (LBNL); 2014.
66. Kim D, Paggi J M, Park C, Bennett C, Salzberg S L. Graph-based genome alignment and genotyping with HISAT2 and HISAT-genotype. Nature Biotechnology. 2019, 37(8):907-15.
67. Liao Y, Smyth G K, Shi W. FeatureCounts: An efficient general-purpose program for assigning sequence reads to genomic features. Bioinformatics. 2014, 30(7):923-30.
68. Love M I, Huber W, Anders S. Moderated estimation of fold change and dispersion for RNA-seq data with DESeq2. Genome Biology. 2014, 15(12):1-21.
69. Virtanen P, Gommers R, Oliphant T E, Haberland M, Reddy T, Cournapeau D, et al. SciPy 1.0: fundamental algorithms for scientific computing in Python. Nature Methods. 2020, 17(3):261-72.
70. Seabold S, Perktold J. Statsmodels: Econometric and Statistical Modeling with Python. In: Proceedings of the 9^{th} Python in Science Conference 2010 Jun. 28 (Vol. 57, No. 61, pp. 10-25080).
71. Grabherr M G, Haas B J, Yassour M, Levin J Z, Thompson D A, Amit I, et al. Nature Biotechnology. 2011, 29(7):644-52.
72. Perkel J M. The software that powers scientific illustration. Nature. 2020, 582(7810):137-9.
73. Barros K O, Mader M, Krause D J, Pangilinan J, Andreopoulos B, Lipzen A, Mondo S J, Grigoriev I V, Rosa C A, Sato T K, Hittinger C T. Oxygenation influences xylose fermentation and gene expression in the yeast genera Spathaspora and Scheffersomyces. Biotechnol Biofuels Bioprod. 2024 Feb. 7; 17(1):20.

What is claimed is:

1. A recombinant yeast comprising one or more recombinant genes, wherein the one or more recombinant genes are configured to express one or more enzymes, wherein the one or more enzymes comprise any one or more of:
a xylose reductase comprising a sequence at least 90% identical to SEQ ID NO:1;
a xylitol dehydrogenase comprising a sequence at least 90% identical to SEQ ID NO:3; and
a xylulokinase comprising a sequence at least 90% identical to SEQ ID NO:5.

2. The recombinant claim 1, wherein the recombinant yeast is of a species unable to consume xylose in native form and wherein the recombinant yeast is capable of consuming xylose.

3. The recombinant yeast of claim 1, wherein the recombinant yeast is from the genus Saccharomyces.

4. The recombinant yeast of claim 1, wherein the recombinant yeast is Saccharomyces cerevisiae.

5. The recombinant yeast of claim 1, wherein:
the xylose reductase, if present, comprises a sequence at least 95% identical to SEQ ID NO:1;
the xylitol dehydrogenase, if present, comprises a sequence at least 95% identical to SEQ ID NO:3; and
the xylulokinase, if present, comprises a sequence at least 95% identical to SEQ ID NO:5.

6. The recombinant yeast of claim 1, wherein:
the xylose reductase, if present, comprises a sequence at least 99% identical to SEQ ID NO:1;
the xylitol dehydrogenase, if present, comprises a sequence at least 99% identical to SEQ ID NO:3; and
the xylulokinase, if present, comprises a sequence at least 99% identical to SEQ ID NO:5.

7. The recombinant yeast of claim 6, wherein the recombinant yeast is Saccharomyces cerevisiae.

8. The recombinant yeast of claim 1, wherein the one or more recombinant genes are configured to express the xylose reductase.

9. The recombinant yeast of claim 1, wherein the one or more recombinant genes are configured to express the xylitol dehydrogenase.

10. The recombinant yeast of claim 1, wherein the one or more recombinant genes are configured to express the xylose reductase and the xylitol dehydrogenase.

11. The recombinant yeast of claim 10, wherein the recombinant yeast is of a species unable to consume xylose in native form and wherein the recombinant yeast is capable of consuming xylose.

12. The recombinant yeast of claim 10, wherein the recombinant yeast is from the genus Saccharomyces.

13. The recombinant yeast of claim 10, wherein the recombinant yeast is Saccharomyces cerevisiae.

14. The recombinant yeast of claim 13, wherein the one or more recombinant genes are configured to express the xylulokinase.

15. The recombinant yeast of claim 13, wherein:
the xylose reductase comprises a sequence at least 95% identical to SEQ ID NO:1; and
the xylitol dehydrogenase comprises a sequence at least 95% identical to SEQ ID NO:3.

16. The recombinant yeast of claim 15, wherein, the recombinant yeast is capable of consuming xylose under anaerobic conditions.

17. The recombinant yeast of claim 15, wherein the recombinant yeast is capable of growing, consuming xylose, and/or producing ethanol in a culture medium consisting of 1% w/v yeast extract, 2% w/v peptone, 2% w/v xylose, and water under anaerobic conditions.

18. The recombinant yeast of claim 15, wherein, the recombinant yeast is capable of producing ethanol in a culture medium consisting of 1% w/v yeast extract, 2% w/v peptone, 2% w/v xylose, and water under anaerobic conditions.

19. The recombinant yeast of claim 15, wherein the recombinant yeast exhibits enhanced growth, enhanced xylose consumption, and/or enhanced ethanol production in a culture medium consisting of 1% w/v yeast extract, 2% w/v peptone, 2% w/v xylose, and water under anaerobic conditions with respect to a control yeast lacking the xylose reductase, the xylitol dehydrogenase, and the xylulokinase and comprising:
a control xylose reductase comprising a sequence of SEQ ID NO:7;
a control xylitol dehydrogenase comprising a sequence of SEQ ID NO:8; and
a control xylulokinase comprising a sequence of SEQ ID NO:9.

20. A method of consuming xylose, the method comprising culturing the recombinant yeast of claim 1 in a culture medium comprising xylose over a time period, wherein the recombinant yeast consumes at least some of the xylose over the time period.
